(19) United States
(12) Patent Application Publication
(10) Pub. No.: US 2012/0323221 A1
(43) Pub. Date: Dec. 20, 2012

(54) Adapter Cap and Nasal Washing System Using the Cap

(75) Inventors: David Gallo, San Diego, CA (US); Jeff Harlan, Corona, CA (US); Lisa Franklin, Milwaukee, WI (US)

(73) Assignee: MED-SYSTEMS, INC., Madison, WI (US)

(21) Appl. No.: 13/418,047

(22) Filed: Mar. 12, 2012

Related U.S. Application Data
(60) Provisional application No. 61/498,787, filed on Jun. 20, 2011.
(60) Provisional application No. 61/569,049, filed on Dec. 9, 2011.

(51) Int. Cl. A61M 3/02

(52) U.S. Cl. 604/514; 604/181

(57) ABSTRACT

An adapter cap is configured to convert a plastic water or drinking bottle into a nasal washing device. The cap has a connector portion or mechanism configured for engagement with the neck of a plastic water bottle in place of a conventional bottle cap, and a tapered applicator or spout portion having a tip with an opening which is configured for placing into a nostril of a user during a nasal washing procedure.

### RELATED APPLICATION

The present application claims the benefit of co-pending U.S. Provisional pat. App. Ser. No. 61/498,787 filed Jun. 20, 2011, and also claims the benefit of co-pending U.S. Provisional pat. App. Ser. No. 61/569,049 filed on Dec. 9, 2011, and the contents of the foregoing applications are incorporated herein by reference in their entirety.

### BACKGROUND

1. Field of the Invention

The present invention is in the field of devices and methods for washing nasal passages and sinuses.

2. Related Art

Nasal washes have been used in the past to deliver a saline solution to an adult user's nasal passages (through a user's nostrils) to wash or lavage the nasal passages to relieve symptoms caused by sinus infections, allergies and the common cold, including nasal congestion, irritated nasal passages, nasal drainage, post-nasal drip, cough, and nasal headaches. A number of devices have been developed for delivering a saline solution to the nasal passages, such as the so-called Neti pot as well as squeeze bottle nasal irrigation devices. These can be difficult to clean and also take up a significant amount of storage room on bathroom shelves and the like.

A Neti pot looks like a small teapot with an extended spout. The pot is filled with a mixture of lukewarm water and salt. The user then tilts their head over a sink at an angle, with one side of the face lower than the other. The spout of the Neti pot is inserted into the upper nostril and the salt solution is allowed to flow into the nostril. The solution flows through the nasal cavity and into the lower nostril, then out of the nostril into the sink. This washes the nasal cavities and helps to thin the mucus, which may ease congestion.

It can be inconvenient to carry a Neti pot when traveling, at work, or away from home for other reasons. Thus, there is a need for a more convenient nasal washing system which can be readily carried.

### SUMMARY OF THE INVENTION

According to one embodiment, an adapter cap is provided which can convert a plastic water bottle into a nasal washing device, and which has an applicator tip having an opening which is configured for placing into a nostril during a nasal washing procedure. The cap is configured for attachment to the neck of a plastic water bottle. In one embodiment, the cap has internal partial threads configured for mating engagement with the threads on the neck of a plastic water bottle. In other embodiments, the cap has retaining tabs for hooking over the annular collar on a bottle neck, or has a retention plug or collar designed for sealing engagement inside the neck of a plastic water bottle. In one embodiment, the cap may be designed to fit more than one size of standard plastic water bottle.

In one embodiment, the cap is made of relatively inexpensive plastic material. A package or portable container containing a plurality of adapter caps and packets of pharmaceutical grade salt (or other type of salt) may be provided. This can be conveniently stored at home and takes up less space than a conventional nasal irrigation device such as a Netipot, and has the added advantage that it is lightweight and compact for the user to carry with them when traveling, at work, or away from home. In order to wash the nasal cavity, the user simply takes a plastic water bottle, removes the existing cap, pours a package of salt into the water, then attaches the adapter cap to the neck of the bottle, then shakes the bottle to dissolve the salt. The tapered spout or applicator tip of the cap is then placed in the nostril with the user tilting their head over a sink so that the saline solution can flow through the nasal cavities and out of the other nostril. The procedure may be repeated with the applicator tip inserted in the opposite nostril. The water bottle can be recycled or discarded when the bottle is empty. The cap may be discarded or recycled when the water bottle is empty, or may be cleaned for further use.

Since tap water is no longer recommended for use in existing nasal washing devices such as Netipots and squeeze bottle irrigation devices, the adapter cap provides a convenient means allowing bottled water to be used for nasal irrigation, either at home or away from home. Bottled water is generally considered to be safer and cleaner than tap water, and is readily available for purchase from many stores while traveling or away from home. The adapter cap is lightweight, inexpensive and easy to use, and provides a convenient nasal washing system when combined with a standard plastic water bottle. Caps and packets of salt can be stored in the bathroom and carried with the user at all times when away from home, taking up very little space.

Other features and advantages of the present invention will become more readily apparent to those of ordinary skill in the art after reviewing the following detailed description and accompanying drawings.

### BRIEF DESCRIPTION OF THE DRAWINGS

The details of the present invention, both as to its structure and operation, may be gleaned in part by study of the accompanying drawings, in which like reference numerals refer to like parts, and in which:

FIG. 1 is a side elevation view of a first embodiment of an adapter cap attached to the threaded neck of a standard plastic water bottle, which is partially broken away;

FIG. 2 is a cross sectional view on the lines 2-2 of FIG. 1 illustrating engagement between one of the partial threads of the adapter cap of FIG. 1 with the threads of a water bottle;

FIG. 2A is an exploded view of the circled area in FIG. 2, illustrating the partial thread engagement in more detail.

FIG. 3 is an exploded, perspective of the adapter cap and threaded neck of the bottle prior to attachment of the cap to the bottle;

FIG. 4 is a bottom plan view of the adapter cap illustrating the location of the partial threads;

FIG. 5 is a cross-section on the line 5-5 of FIG. 4, illustrating the staggered height of two of the partial threads;

FIG. 6 is a perspective view of the upper end of a bottle with the cap of FIGS. 1 to 5 separate from the bottle, illustration the addition of salt to the bottle prior to attachment of the cap;

FIG. 7 is a side elevational view illustrating a second embodiment of an adapter cap attached to the neck of a standard water bottle, with the lower portion of the bottle broken away;

FIG. 8 is a top plan view of the cap of FIG. 7 and broken away bottle;

FIG. 9 is a cross-sectional view on the lines 9-9 of FIG. 8;

FIG. 9A is an exploded view of the circled area in FIG. 9, illustrating the engagement between one of the partial threads on the cap with the threads on the bottle neck;

FIG. 10 is an exploded, perspective of the adapter cap of FIGS. 7 to 9 and threaded neck of the bottle prior to attachment of the cap to the bottle;

FIG. 11 is a bottom plan view of the cap of FIGS. 7 to 10 illustrating the location of the three partial threads in the cap;

FIG. 12 is a cross sectional view on the lines 12-12 of FIG. 11;

FIG. 13 is an exploded perspective view of a two part adapter cap according to another embodiment aligned with the neck of a standard plastic water bottle prior to attachment to the bottle;

FIG. 14 is a side elevation view illustrating the two part cap of FIG. 13 assembled and attached to the neck of the bottle;

FIG. 15 is a top plan view of the two part cap of FIGS. 13 and 14 attached to the bottle;

FIG. 16 is a cross-sectional view on the lines 16-16 of FIG. 15;

FIG. 17 is a bottom perspective view of a modified adapter cap which combines the two parts of the cap of FIGS. 13 to 16 into a one part, sealing adapter cap;

FIG. 18 is a cross-sectional view similar to FIG. 16 but illustrating the one part sealing adapter cap secured to the neck of a bottle;

FIG. 19 is an exploded, perspective view of a two part adapter cap according to another embodiment aligned with the neck of a bottle prior to attachment to the bottle;

FIG. 20 is a side elevation view illustrating the two part adapter cap of FIG. 19 attached to the neck of a bottle;

FIG. 21 is a cross-sectional view on the lines 21-21 of FIG. 20;

FIG. 22 is a bottom plan view of a first part of the adapter cap of FIGS. 19 to 21;

FIG. 23 is a front perspective view of an adapter cap according to another embodiment;

FIG. 24 is a bottom perspective view of the adapter cap of FIG. 23 and the neck of a bottle shown partially broken away, prior to attachment of the cap to the neck of the bottle;

FIG. 25 is a side elevation view illustrating the cap of FIGS. 23 and 24 attached to the bottle;

FIG. 26 is a top plan view illustrating the cap attached to the bottle as in FIG. 25;

FIG. 27 is a cross-sectional view on the lines 27-27 of FIG. 26;

FIG. 28 is a front perspective view of an adapter cap according to another embodiment which is a modification of the embodiment of FIGS. 1 to 6;

FIG. 29 is a bottom perspective view of the adapter cap of FIG. 28;

FIG. 30 is a side elevation view of the cap of FIGS. 28 and 29;

FIG. 31 is a top plan view of the cap of FIGS. 28 to 30;

FIG. 32 is a cross-sectional view of the cap on the lines 32-32 of FIG. 30;

FIG. 32A is an enlarged view of the circled area A of FIG. 32;

FIG. 33 is a cross-sectional view of the cap on the lines 33-33 of FIG. 31;

FIG. 33A is an enlarged view of the circled area N in FIG. 33;

FIG. 34 is a front perspective view of an adapter cap according to another embodiment which is a modification of the embodiment of FIGS. 28 to 33A;

FIG. 35 is a bottom perspective view of the cap of FIG. 34;

FIG. 36 is a side elevation view of the cap of FIGS. 34 and 35;

FIG. 37 is a top plan view of the cap of FIGS. 34 to 36;

FIG. 38 is a cross-sectional view of the cap on the lines 38-38 of FIG. 36;

FIG. 39 is a cross-sectional view of the cap on the lines 39-39 of FIG. 37;

FIG. 40 is a front perspective view of an adapter cap including an air return according to another embodiment;

FIG. 41 is a top plan view of the cap of FIG. 40;

FIG. 42 is a bottom plan view of the cap of FIG. 40;

FIG. 43 is a cross-sectional view on the lines 43-43 of FIG. 41;

FIG. 44 is a perspective view illustrating the lower end of the cap of FIGS. 40-43;

FIG. 45 is a perspective view similar to FIG. 40 illustrating a modification of the air return of the cap of FIGS. 40 to 44;

FIG. 46 is a cross sectional view similar to FIG. 43 illustrating the modified air return check valve;

FIG. 47 is a bottom perspective view similar to FIG. 44 but illustrating the modified air return with check valve;

FIG. 48 is a front perspective view of an adapter cap according to another embodiment;

FIG. 49 is a top plan view of the cap of FIG. 48;

FIG. 50 is a side elevation view of the cap of FIGS. 48 and 49;

FIG. 51 is a cross-sectional view on the lines 51-51 of FIG. 50;

FIG. 51A is an enlarged view of the circled area A of FIG. 51;

FIG. 52 is a cross-sectional view on the lines 52-52 of FIG. 49, illustrating the cap engaged on the bottle neck of a first type of water bottle;

FIG. 53 is a view similar to FIG. 52 but illustrating the cap engaged on a larger diameter bottle neck of a second type of water bottle;

FIG. 54 is a front perspective view of a modified version of the adapter cap of FIGS. 34 to 39;

FIG. 55 is a bottom perspective view of the adapter cap of FIG. 54;

FIG. 56 is a top plan view of the cap of FIGS. 54 and 55;

FIG. 57 is an exploded view of parts of an adapter cap assembly according to another embodiment;

FIG. 58 is a perspective view of the parts of FIG. 57 assembled together for attachment to the neck of a water bottle;

FIG. 59 is a side elevation view of the assembled adapter cap of FIG. 58;

FIG. 60 is a cross-sectional view on the lines 60-60 of FIG. 59;

FIG. 61 is a bottom plan view of the adapter sleeve of FIG. 58;

FIG. 62 is a perspective view illustrating a modified version of the adapter cap of FIGS. 34 to 39 with a protective cover, illustrating the cap separated from a bottle neck;

FIG. 63 is a side elevation view of the cap of FIG. 62, illustrating the cap attached to a bottle neck;

FIG. 64 is a top plan view of the adapter cap of FIGS. 62 and 63;

FIG. 65 is a cross-sectional view on the lines 65-65 of FIG. 64;

FIG. 66 is an enlarged view of the circled area B of FIG. 65;

FIG. 67 is a bottom plan view of the adapter cap of FIGS. 62 to 66; and

FIG. 68 is a side elevation view of the adapter cap of FIGS. 62 to 67 with the cover separated from the cap to uncover the opening.

### DETAILED DESCRIPTION

Certain embodiments as disclosed herein provide for an adapter cap for a nasal washing system, and a nasal washing system or kit including one or more caps and packets of pharmaceutical grade saline.

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention.

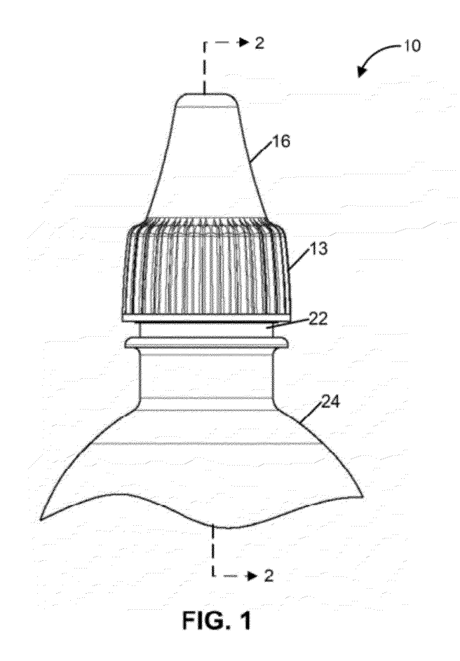

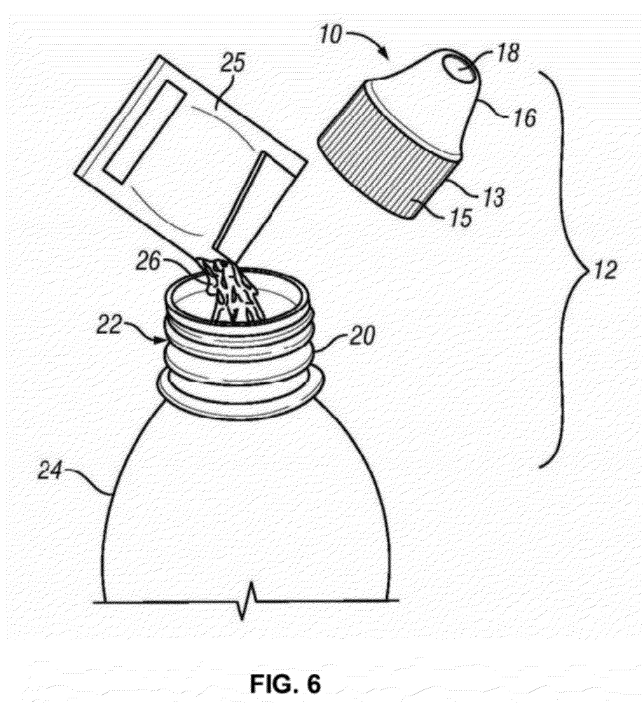

FIGS. 1 to 5 illustrate a first embodiment of an adapter cap 10 which may be used as part of one embodiment of a nasal washing system 12 as illustrated in FIG. 6. Cap 10 may be formed from molded plastic material or the like, and in one embodiment has a polypropylene injection molded body. Cap 10 has a connector or neck portion 13 having an external gripping surface 15 and offset partial threads on its inner surface 17, and a tapered spout portion 16 extending from connector portion 13 and having a dispensing opening 18 at the end or tip of the spout. In this embodiment, the entire spout portion 16 is of generally frusto-conical shape up to the applicator tip or end of portion 16, but in other embodiments only part of the spout extending up to the tip may be of frusto-conical shape. The offset partial threads comprise three spaced partial threads 14A, 14B, 14C which are designed for mating engagement with the external threads 20 on the neck 22 of a plastic water bottle 24, which is shown partially broken away in FIGS. 1, 2, 3 and 6. Although one example of a standard plastic water bottle 24 is illustrated in FIG. 1, it should be understood that cap 10 can be attached to other US standard water bottles of different shapes and sizes in a similar manner, and can engage with different types of external threads. In one embodiment, cap 10 is designed to fit most 26-28 mm. plastic water bottles.

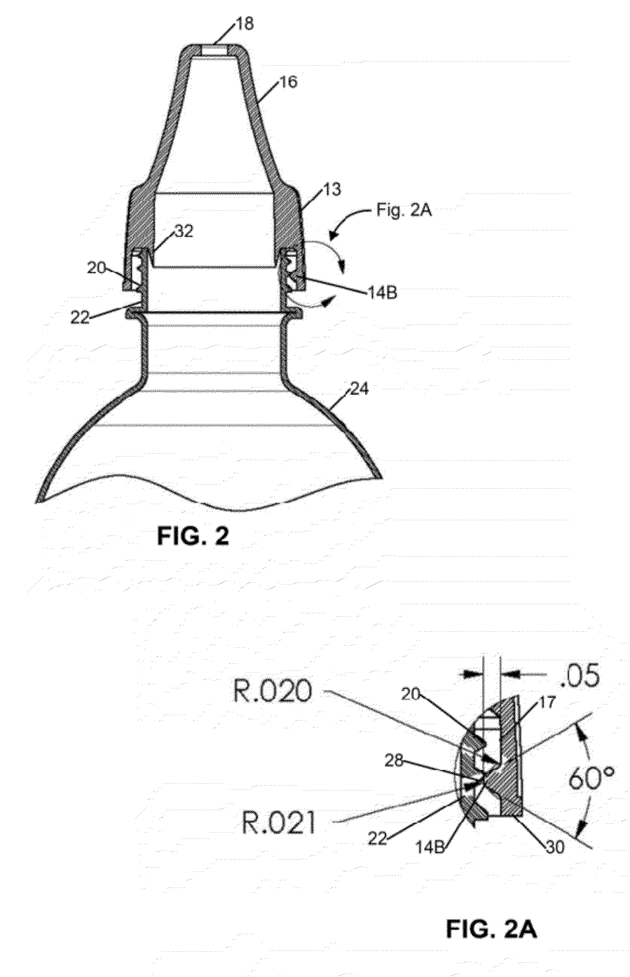

As illustrated in FIG. 2, connector portion 13 is generally cylindrical and has an annular wall of reduced thickness having inner surface 17 in a lower end part of the cap, and a thicker wall section extending up to spout portion 16. The thicker section of the wall has a downwardly projecting annular rim 32 which is configured to engage inside the neck 22 of a plastic water bottle 24, as illustrated in FIG. 2.

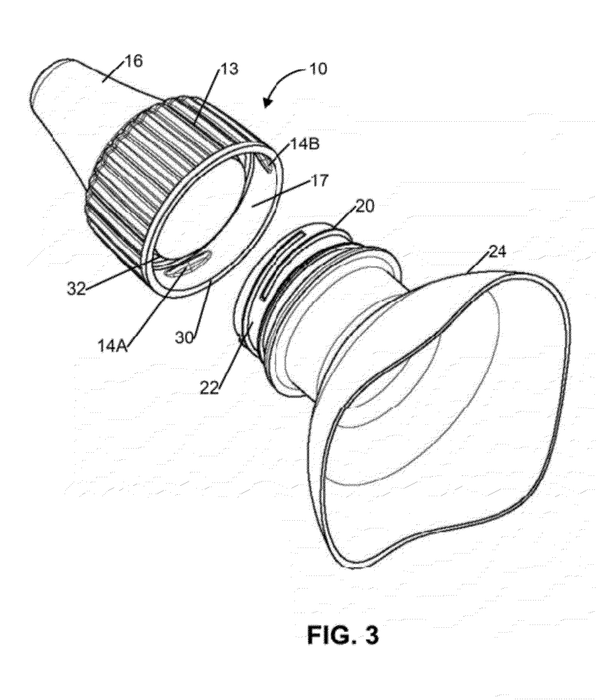

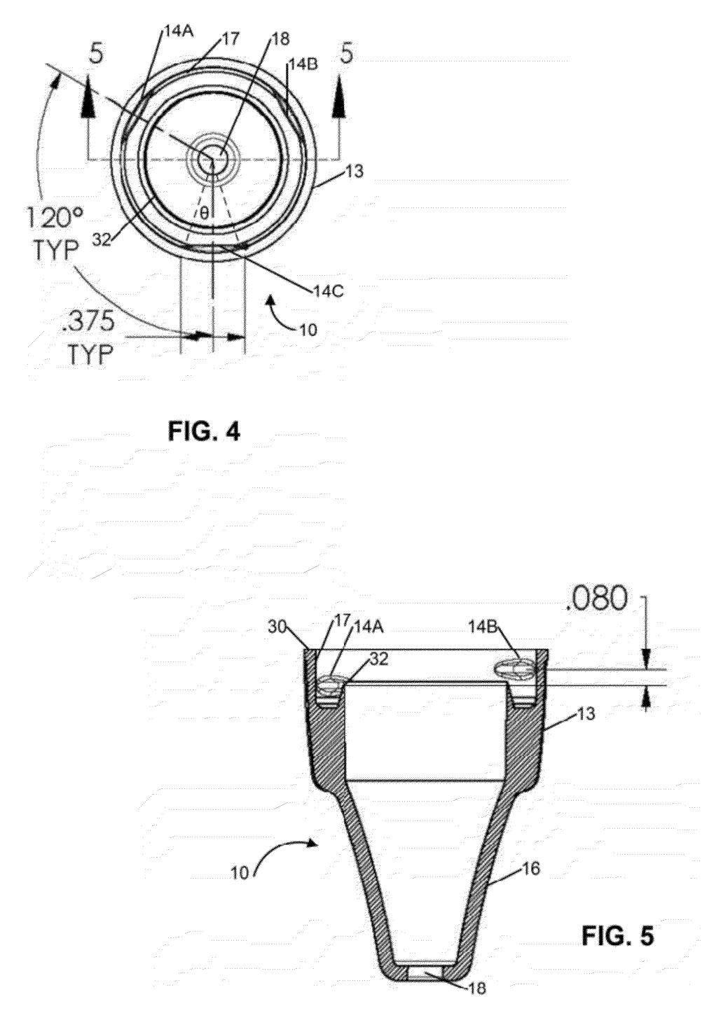

FIGS. 1 and 2 illustrate cap 10 attached to the neck of the water bottle and ready to use for washing a person's nasal cavities, while FIGS. 4 and 5 illustrate the cap separate from the bottle, and FIG. 3 illustrates the cap prior to attachment to an aligned water bottle. As illustrated in FIGS. 2A and 3 to 5, three partial threads 14A to 14C are located at equal angular spacings of 120 degrees about the periphery of the inner surface. Each partial thread is elongated in a circumferential direction and is generally V-shaped in transverse cross section with a rounded or flat apex 28, as illustrated in FIG. 2A, with a height of approximately 0.05 inches and an angle of about 60 degrees at the apex. The partial threads are each of shape similar to a circular segment in longitudinal cross section or end-elevational view, as seen in FIG. 4, and each partial thread subtends an angle θ of around 35 degrees. At least one of the partial threads 14A is offset in height relative to threads 14B and 14C which are located closer to the rim 30 of the cap, and is at a vertical center-to-center spacing of around 0.080 inches from partial thread 14B, as seen in FIG. 5. The partial threads are designed to engage between the threads 20 on the bottle neck as the cap is screwed on, and releasably secure the cap on the bottle when attached as in FIG. 2. A greater or lesser number of offset partial threads may be provided in alternative embodiments. When the adapter cap is secured to bottle 25 as in FIG. 2, the inner end of the annular groove between annular rim 32 and wall surface 17 forms a seal with the upper rim or lip of bottle neck 22.

FIG. 6 illustrates the separated components of a nasal washing system 12 comprising adapter cap 10, a packet 25 of salt or sodium chloride 26 as generally used for washing the nasal cavities, and a standard plastic water bottle 24. The contents of the packet may be sodium chloride alone, or sodium chloride mixed with other materials suitable for nasal washing purposes. In one embodiment, the dry particulate or powder material 26 is a preservative free, pharmaceutical grade salt (or other type of salt). In FIG. 6, the existing cap of water bottle 24 has been removed, and a user is pouring the contents 26 of packet 25 into the water bottle for mixing with the water in the bottle to form a saline solution. Once the packet is emptied or sufficient salt has been added to the water, and mixed with the water to form a saline solution, the adapter cap is secured to the top of the bottle as illustrated in FIGS. 1 and 2. The bottle can be shaken with the original cap attached in order to dissolve the salt, or can be shaken after attachment of the adapter cap with the user holding their finger or thumb over opening 18. The user then tilts their head to one side over a sink, bucket, or the like, inserts the spout portion or applicator tip at the upper end of tapered portion 16 into the upper nostril so that the frusto-conical tip forms a seal with the rim of the nostril, and allows the saline solution to pour into the nostril, through the nasal cavities, and out through the lower nasal cavity, repeating the procedure with their head tilted to the opposite side. The saline solution dispensed from the bottle through the adapter cap washes the nasal passages to relieve symptoms caused by sinus infections, allergies, flu and the common cold, including nasal congestion, irritated nasal passages, nasal drainage, post-nasal drip, cough, and nasal headaches. This thins mucus, removes inhaled irritants (e.g., dust, pollen, smoke), promotes nasal and sinus drainage, and helps reduce swelling of nasal membranes.

The adapter cap can be re-used or discarded after use. A kit may be provided comprising a container or small canister or envelope containing a plurality of thin, lightweight plastic adapter caps, and a corresponding number of packets containing a measured amount of salt for use in a nasal saline solution, such as pharmaceutical grade salt (or other type of salt). This kit is lightweight and convenient to carry around in a pocket, purse or suitcase, so that a user can easily transport the kit with them while traveling or away from home. All that is needed to complete the system is purchase of a conventional bottle of water, which is readily available in most locations. Alternatively, the nasal wash system may be used to wash tissues other than nasal tissue with a saline solution or a different solution, such as, but not by way of limitation, a mucus membrane, eye tissue, and skin or tissue inside an oral cavity.

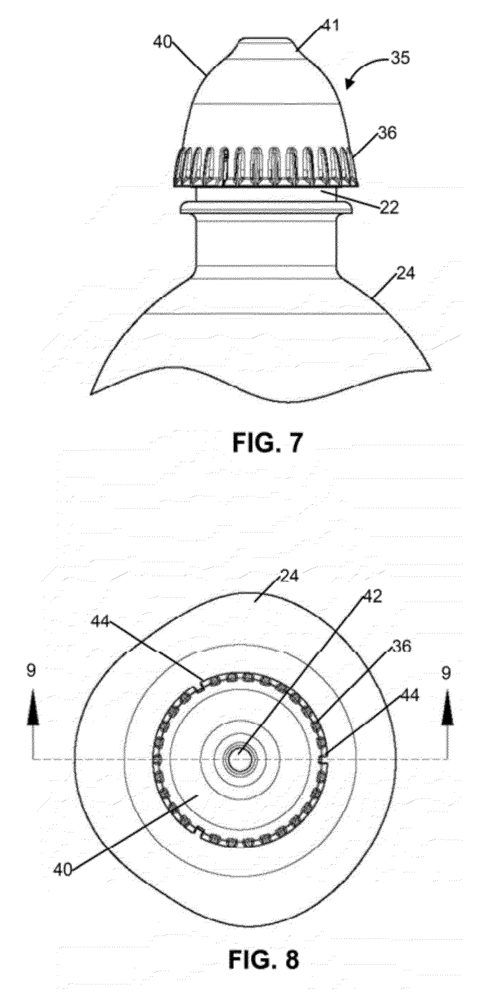

It will be understood that any of the alternative embodiments of the adapter cap described below in connection with FIGS. 7 to 68 may be used in place of cap 10 in the nasal washing system of FIG. 6.

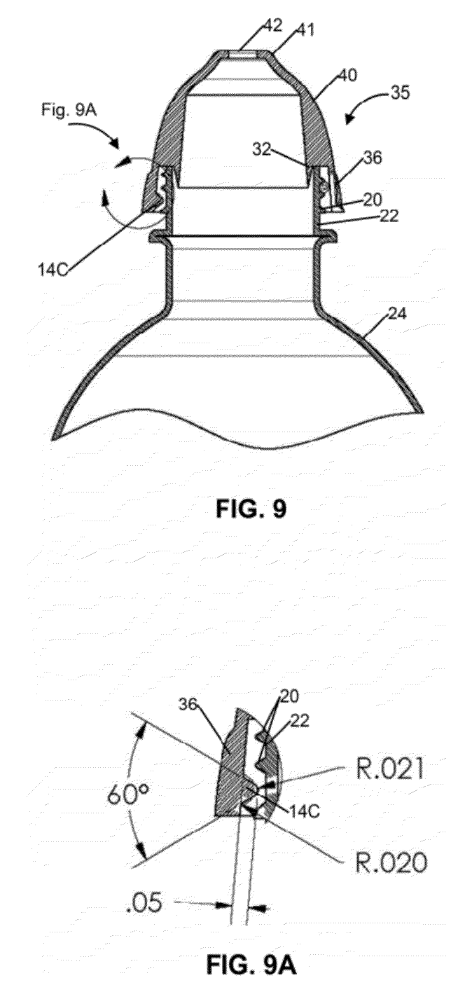

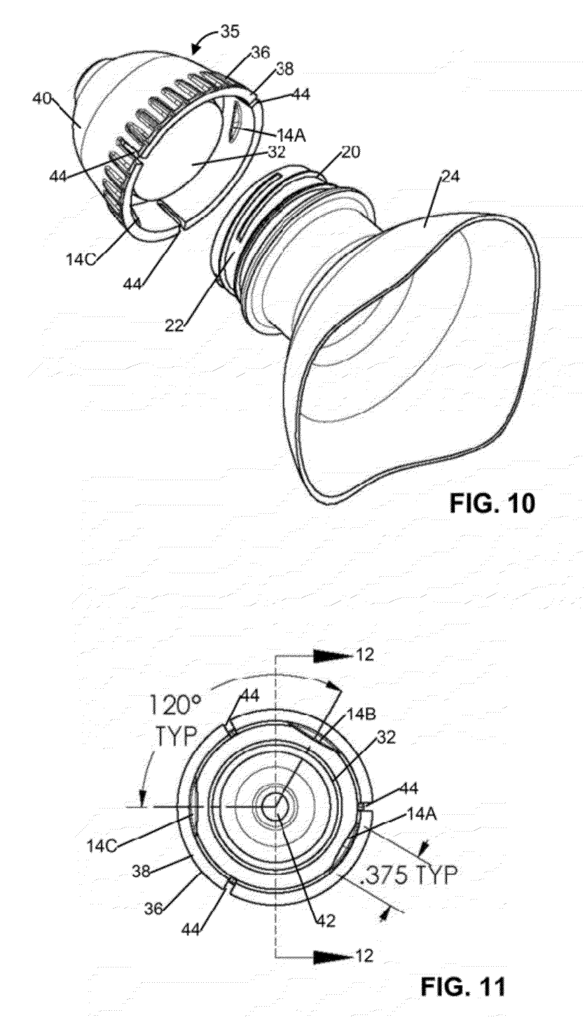

FIGS. 7 to 12 illustrate a second embodiment of an adapter cap 35 which can also be releasably attached to the threaded neck of a standard plastic water bottle, such as water bottle 24 or any other type of water bottle. Cap 35 has a connector portion 36 with partial internal threads designed for releasable threaded engagement with external threads 22 on the neck 20 of a water bottle, as in the previous embodiment, and like reference numbers are used for like parts as appropriate. However, the cap is shorter than the cap of the previous embodiment, and has a shorter external gripping surface and a more rounded, dome-like spout portion 40 extending from connector portion 36 to a tapered applicator tip 41 having a dispensing opening 42 at the upper end of the cap. An annular rim 32 is provided inside the cap to engage inside the neck of a bottle as in the previous embodiment, as illustrated in FIG. 9. The connector portion 36 in this embodiment has three spaced relief slots 44 extending upwardly from lower rim 38 to provide for more flexibility when engaging the cap over different size bottle necks. Apart from the reduced height of the cap 35, the modified external shape, and the addition of relief slots 44, cap 35 is attached to the neck of a bottle in much the same way as described above in connection with the first embodiment, and may be made of the same material.

FIGS. 13 to 16 illustrates a two part adapter cap 50 according to a third embodiment in which the partial threads configured to attach the cap to a bottle in the previous embodiments are replaced with a plug or sealing collar configured for sealing engagement inside the neck of a bottle. Cap 50 comprises a main body 52 and sealing collar 54 configured for releasable attachment to the body. The main body 52 may be formed from molded plastic material or the like, and in one embodiment is a polypropylene injection molded body. Sealing collar 54 may be formed of a rubber-like thermoplastic elastomeric material or other flexible material that is more flexible than the remainder of cap 50. As illustrated in FIGS. 13 and 16, body 52 has a through bore 56 with an upper end opening 58 and comprises an upper spout portion 60 and a stem portion 62 of reduced diameter having spaced annular ribs 64. Spout portion 60 has an outer ribbed gripping surface 65 around its lower end and is of generally rounded, tapering shape from the gripping surface 65 to a tapered applicator tip 85 which extends to upper end opening 58. Collar 54 is generally cylindrical and has a through bore 66 and a series of spaced annular gripping ribs 68 on its outer surface.

Ribs 64 on stem portion 62 are designed for gripping engagement in the through bore 66 of collar 54 to secure the stem portion to the collar, as best illustrated in FIG. 16. Ribs 68 on collar 54 are designed for sealing and gripping engagement with the inner surface 55 of the bottle neck 22 when the cap is secured to a bottle, as also illustrated in FIG. 16. The stem portion 62 and attached collar 54 are forced into the open neck 22 of a bottle in order to attach the cap 50 to the bottle. The bottle is then ready for use in rinsing the nasal cavities with saline solution, as described above in connection with FIG. 6.

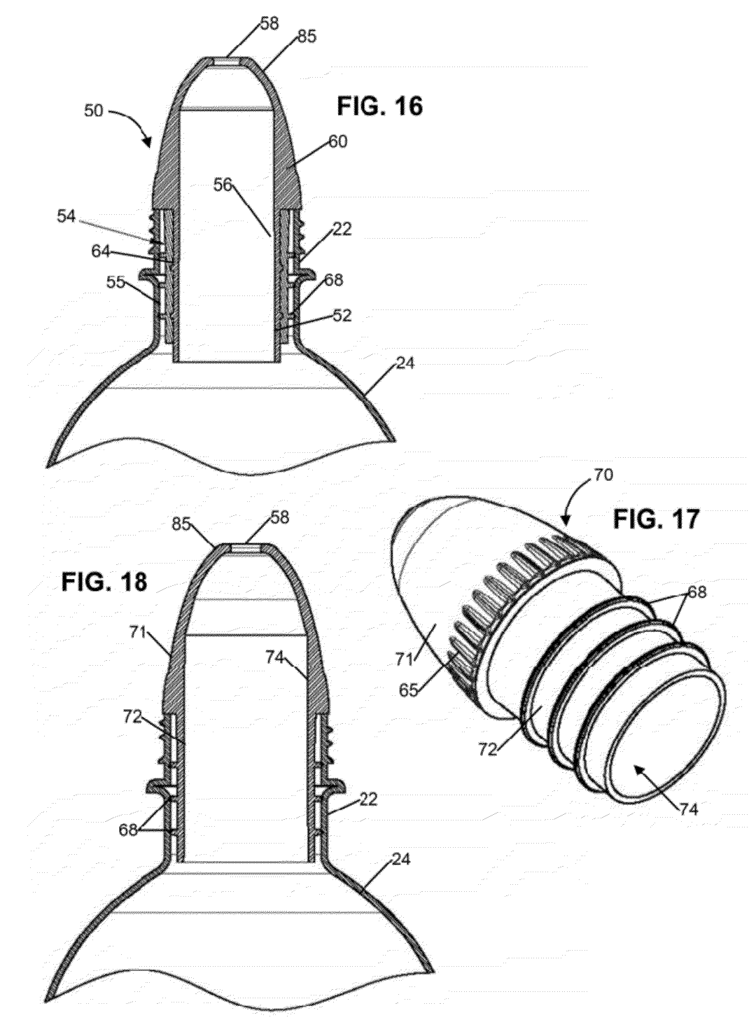

FIGS. 17 and 18 illustrate a modification of the embodiment of FIGS. 13 to 16 in which cap 70 is made in one piece rather than two separate parts. In this embodiment, the entire body of cap 70 is integrally molded of a suitable elastomeric material such as, but not limited to, SYNOPRENE™ or the like, rather than for using the main body separately out of a more rigid material, as in the previous embodiment. Some portions of cap 70 are identical to those of the previous embodiment, and like reference numbers are used for like parts as appropriate.

As in the previous embodiment, cap 70 has an upper spout portion 71 of generally rounded, tapering shape extending up to upper end opening 58, with a gripping portion 65. A reduced diameter collar portion 72 extends downward from the upper body portion, and the cap has a through bore 74 which extends through the entire cap up to opening 58. Collar portion has spaced, annular outer gripping ribs 68, as in the previous embodiment, and engages inside the neck 22 of a bottle as illustrated in FIG. 18 to secure the cap to the bottle.

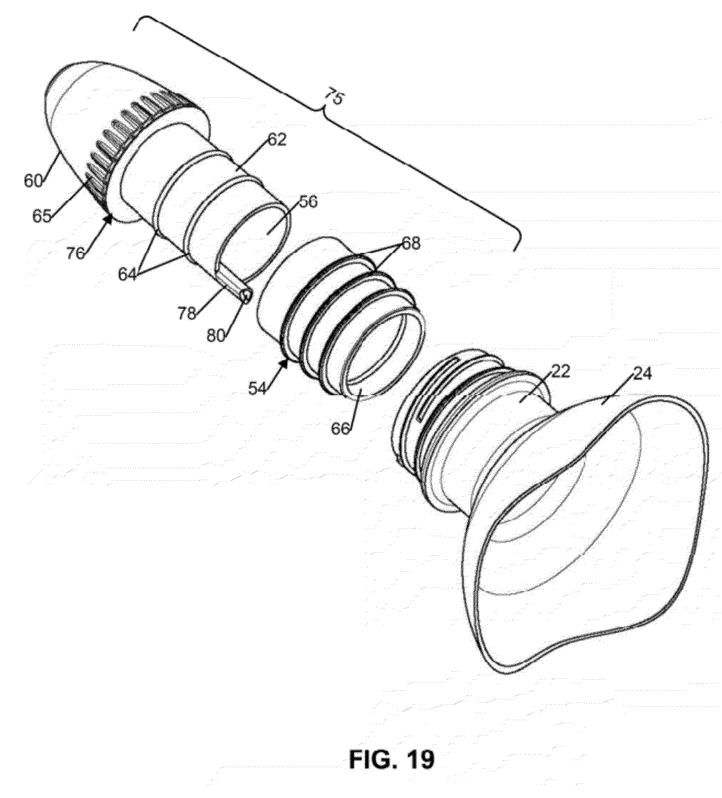

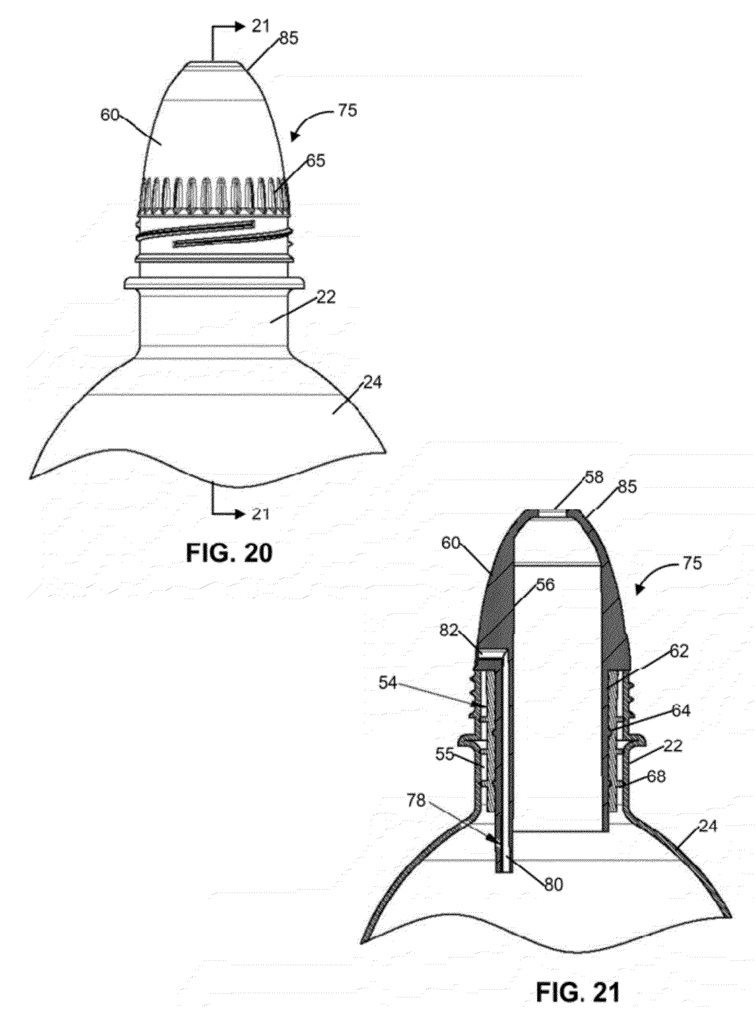

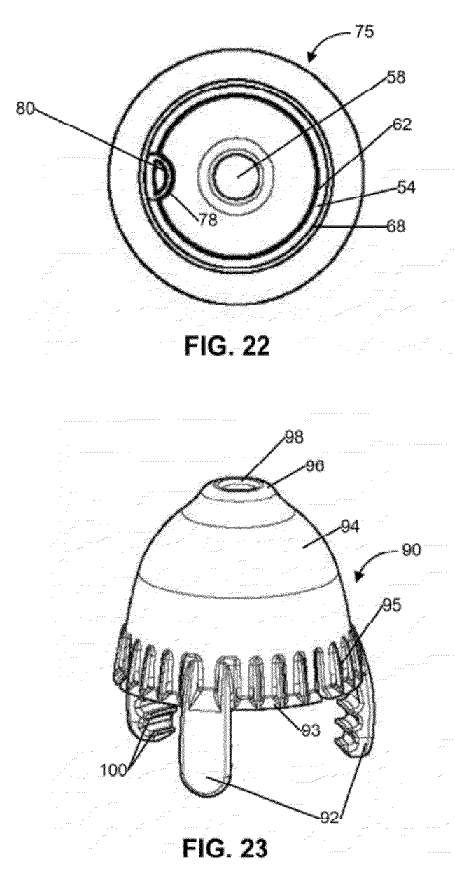

FIGS. 19 to 22 illustrate another embodiment of a two part adapter cap or plug 75 for attachment to a conventional water bottle to allow the bottle and cap to be used for nasal washing. The embodiment of FIGS. 19 to 22 is similar to that of FIGS. 13 to 16, and like reference numbers have been used for like parts as appropriate. As in the embodiment of FIGS. 13 to 16, cap 75 has a main body 76 and a sealing collar 54 configured for releasable attachment to the body and releasable sealing engagement in the neck 22 of a conventional plastic water bottle 24, shown partially broken away in FIGS. 19 to 21. The main body 76 may be formed from molded plastic material or the like, and in one embodiment is a polypropylene injection molded body. Sealing collar 54 may be formed of a rubber-like thermoplastic elastomeric material that is more flexible than the remainder of cap 50.

As in the previous two part adapter cap embodiment of FIGS. 13 to 16, body 76 has a through bore 56 with an upper end opening 58 and comprises an upper spout portion 60 having a tapering applicator portion 85, and a stem portion 62 of reduced diameter having spaced annular ribs 64. However, unlike the previous embodiment, the body 76 also has an integrally formed air intake or inlet tube 78 which protrudes downwardly from stem portion 62. An air intake bore 80 extends through tube 78, through the wall of stem portion 62 into upper portion 60, and then outwardly to an air inlet opening 82 in the outer surface of gripping portion 65. The collar 54 in this embodiment is secured over stem portion 62 via ribs 64, as in the previous two part adapter cap, and the assembled cap is secured in the neck of a bottle in the same way as in the previous embodiment, with the ribs 68 on collar 54 in sealing engagement with the inner surface 55 of bottle neck 22, as illustrated in FIG. 21. The cap of FIGS. 19 to 22 may also be made in one piece in an alternative embodiment, as in FIGS. 17 and 18.

When the cap is attached to a bottle as in FIG. 21, the air intake 78 extends into the bottle and forms a second passageway or air inlet passageway 80 through the opening 82 and through the body of the cap into the bottle. Adapter cap 75 attached to bottle 24 containing saline solution is used in the substantially same way as the cap of the previous embodiments, with the tapering upper end portion or applicator tip 85 inserted into the uppermost nostril with the air opening 82 facing upward. The bottle can be squeezed gently if needed, to increase liquid flow into the nose. As saline solution flows out of the bottle through bore 56 and opening 58, air flows into the bottle through air intake passageway 80.

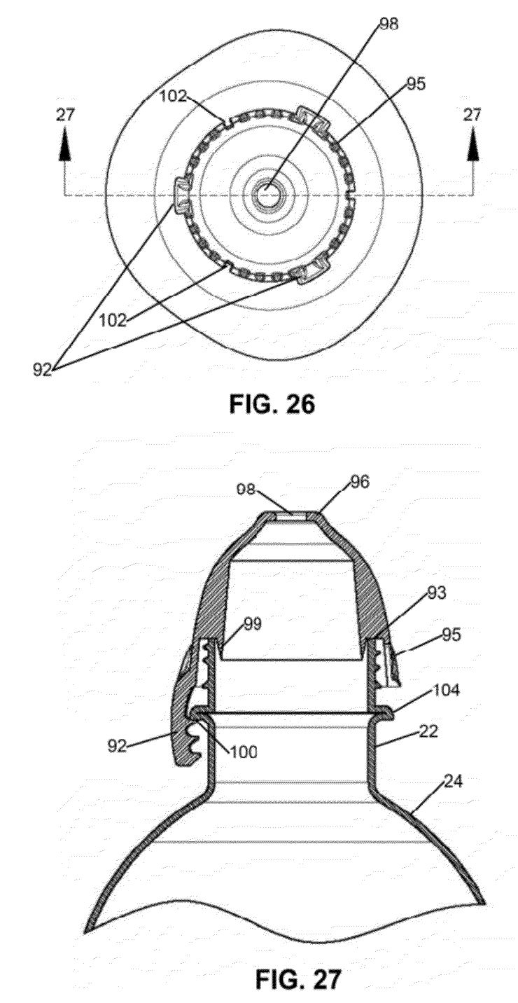

FIGS. 23 to 27 illustrate another embodiment of an adapter cap 90 designed for attachment to the neck of a standard plastic water bottle 24, as illustrated in FIG. 27. In this embodiment, the attachment mechanism is different from the previous embodiments and comprises a series of spaced retaining tabs 92 extending from the lower rim 93 of the cap. The cap is of similar shape to the previous three embodiments and has a lower, annular connector portion 95 and a rounded, tapering spout portion extending from connector portion 95 to applicator tip 96 which has a central opening 98. A downwardly projecting annular rim 99 inside connector portion 95 is designed to engage inside the neck of the bottle, as in the first embodiment. Each retaining tab 92 has a series of three spaced, inwardly projecting teeth 100.

Cap 90 is formed from molded plastic material or the like, and in one embodiment has a polypropylene injection molded body 94. As in the embodiment of FIGS. 7 to 11, a series of three spaced relief slots 102 extend from the lower rim 93 of the cap body to allow more flexibility when attaching to various different bottle necks. Slots 102 are offset from the retaining tabs 92.

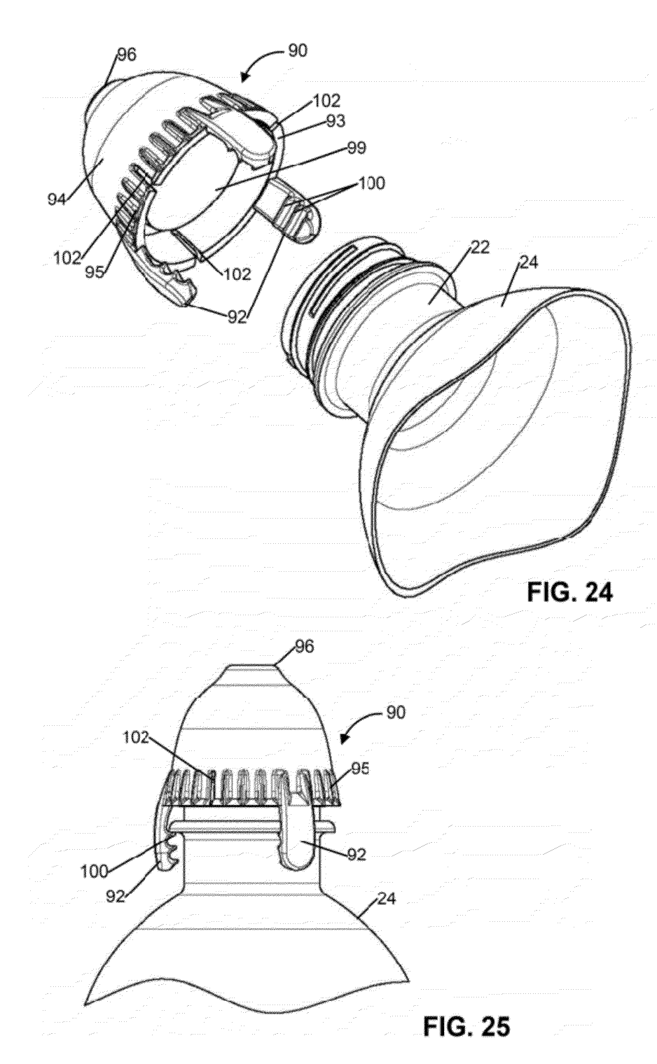

FIG. 24 illustrates cap 90 aligned with the neck 22 of a bottle 24 prior to attachment to the bottle. As the cap is moved into engagement with neck 22, annular rim 99 extends into the open end of the bottle while flexible retaining tabs 92 snap over the annular collar 104 of the bottle neck, with one of the teeth 100 on each tab 92 hooking over the collar to hold the cap in place, as best illustrated in FIGS. 25 and 27. The relief slots 102 allow the tabs 92 to expand outwardly and pass over collar 104. By providing three spaced retention teeth at different heights, the cap can be secured to various different bottles which have collars at different heights on neck 22. The cap is forced down over the bottle neck until the lower rim 93 of the cap engages the upper end of the bottle neck with the annular rim 99 engaging inside the bottle neck, as seen in FIG. 27.

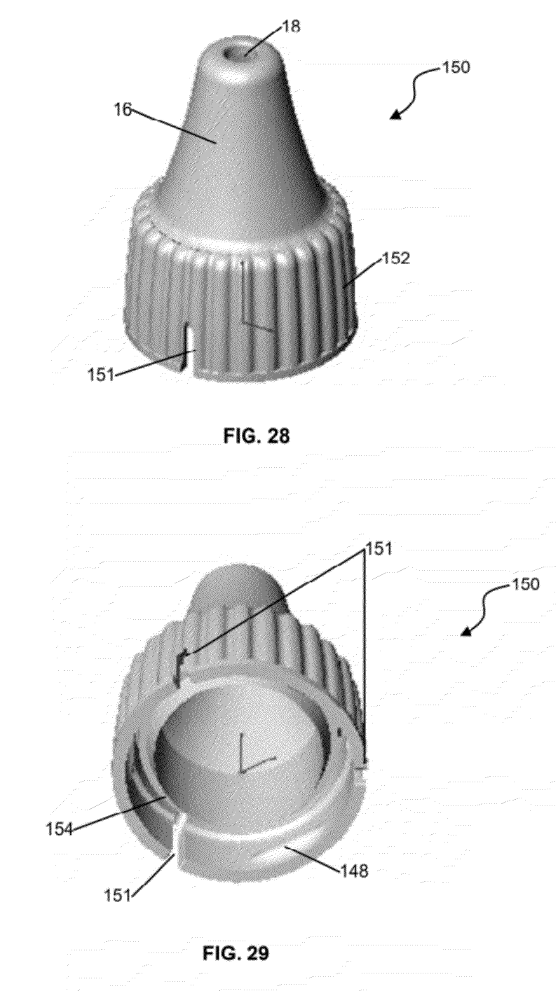

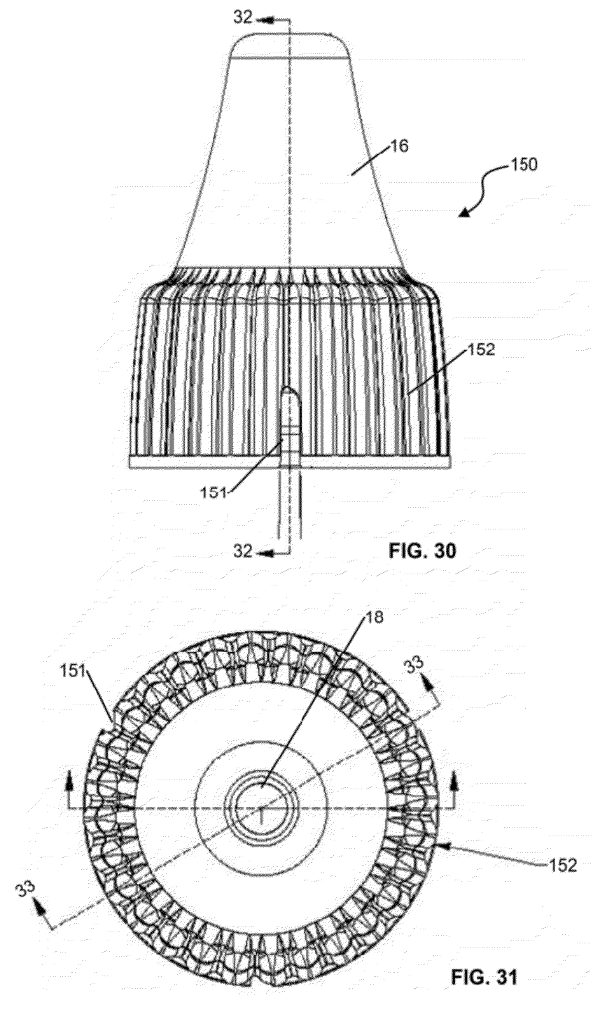

FIGS. 28 to 33 illustrate another embodiment of an adapter cap 150 which is similar to the embodiment of FIGS. 1 to 6 but which includes slits or relief slots 151 similar to the relief slots of the second embodiment in the sides of lower connector portion 152. The upper portion of the cap is similar or identical to that of cap 10 of FIGS. 1 to 6, and like reference numbers are used for like parts as appropriate. Connector portion 152 has an external gripping surface and offset partial threads 14A, 14B, 14C on its inner surface 17, as in cap 10, and a tapered spout portion or generally frusto-conical applicator portion 16 extending from connector portion 152 and having an opening 18 at the tip of the spout. The offset partial threads are designed for mating engagement with the external threads on the neck of a conventional plastic water bottle, and may be capable of fitting the threads of at least some different plastic water bottles, such as most 26 to 28 mm plastic water bottles.

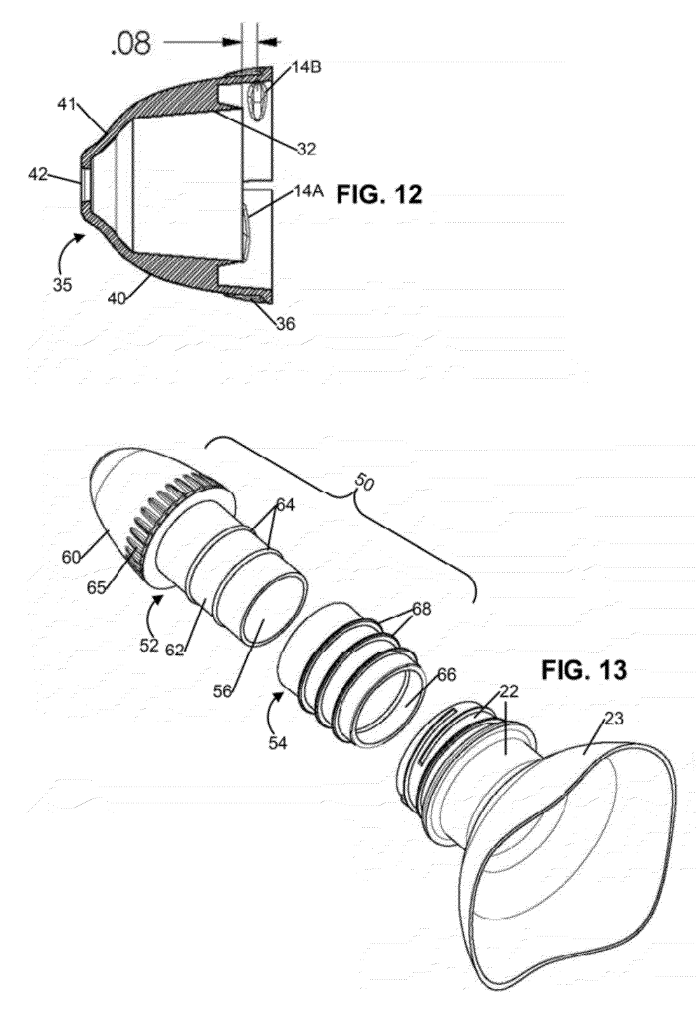

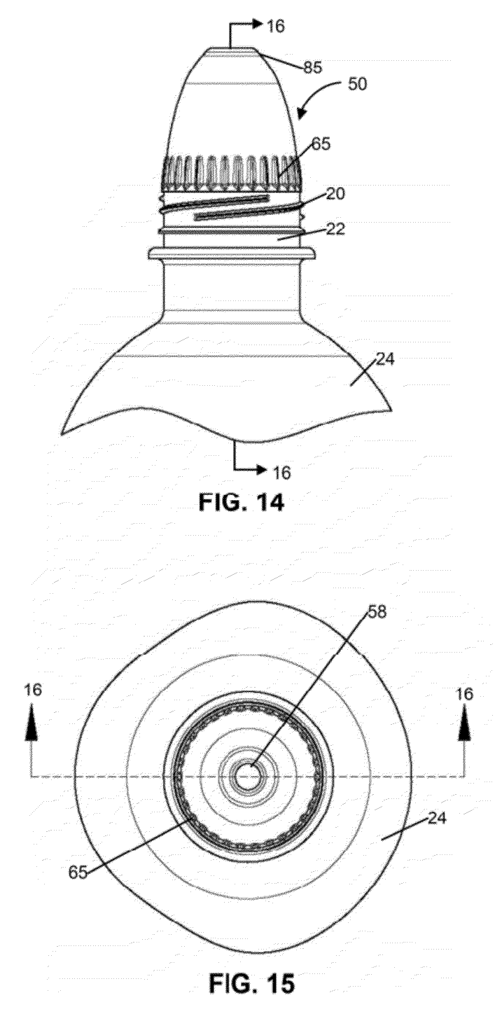

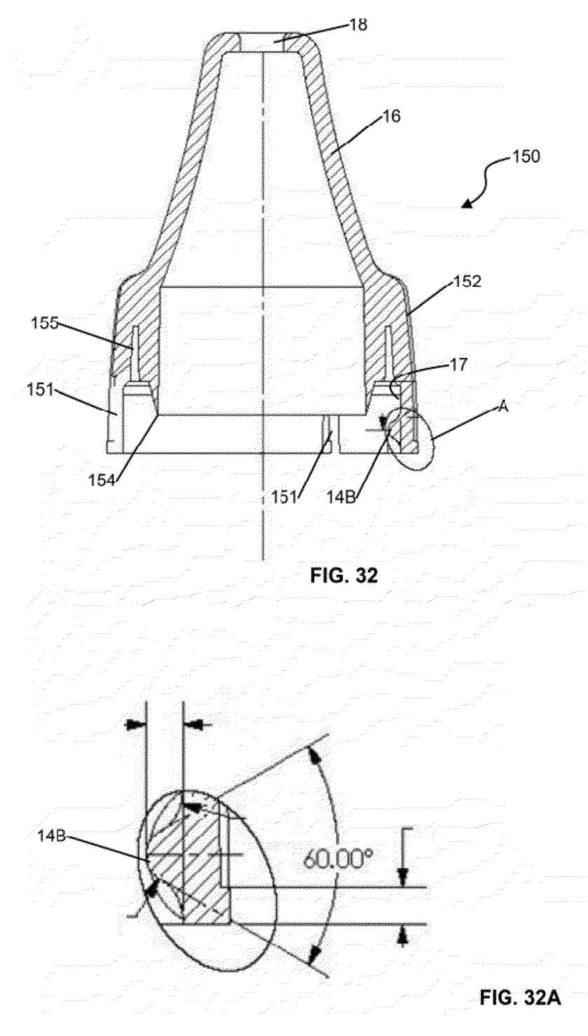

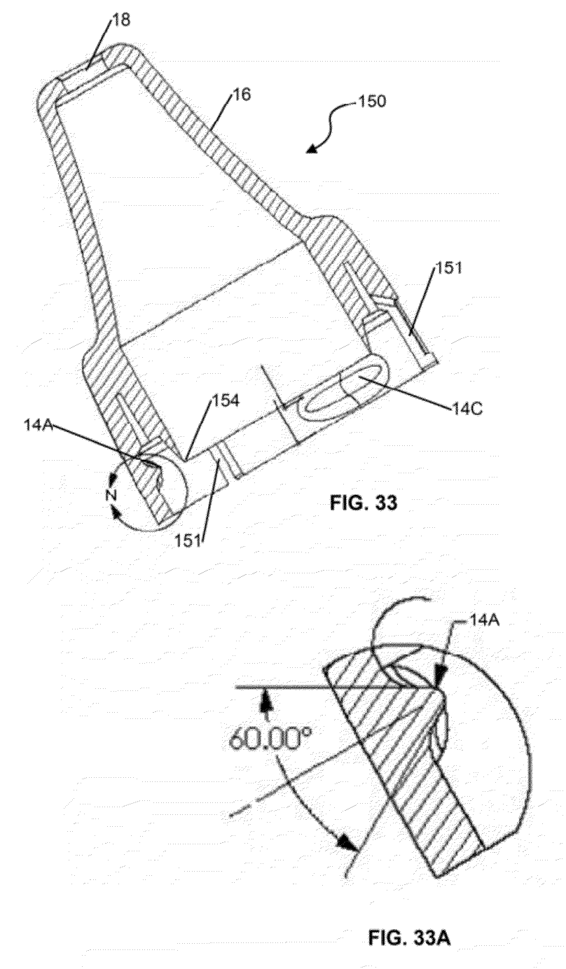

In the illustrated embodiment, a set of three equally spaced slits 151 are spaced around the connector portion, each extending upwardly a predetermined distance from the open lower end of portion 152, as best illustrated in FIGS. 28, 29 and 30. In this embodiment, the height of each slit is around one third to one half of the height of the outer gripping section of portion 152, and the width is around two degrees of the periphery of connector portion 152, or around 1.9 mm in one embodiment. The height of each slit in one embodiment is around 7.5 mm or 0.30 inches. This allows the plastic part to release from the mold more easily, and also allows the cap to fit over different size threads more readily. The internal partial thread formations 14A, 14B and 14C are at similar positions inside connector portion 152 to those of the first embodiment, and may be of the same or slightly different dimensions. In one embodiment, threads 14B (FIG. 32A) and 14C are of slightly larger height and width dimensions to the equivalent threads in the first embodiment, and are around 1.3 mm in height, while the upper thread 14A (FIG. 33A) is of the same dimensions to the first embodiment, and slightly shorter and narrower than the lower threads, with a height of around 1.2 mm. Connector portion 152 also includes a downwardly projecting annular rim 154 which is configured for engagement inside the neck of bottle when the cap is in position, as illustrated in FIG. 6 for the first embodiment Annular groove 155 extends upwardly into the thicker section of the wall of connector portion 152, and allows for more flexing of connector portion 152 as the cap is installed onto a bottle neck.

In one embodiment, the inner surface 17 of connector portion is of the order of one inch or around 27 mm in diameter, while the diameter of annular rim 154 is of the order of 0.8 inches or around 20.4 mm, although caps 150 may be made with different connector portion diameters to fit onto different size bottle necks.

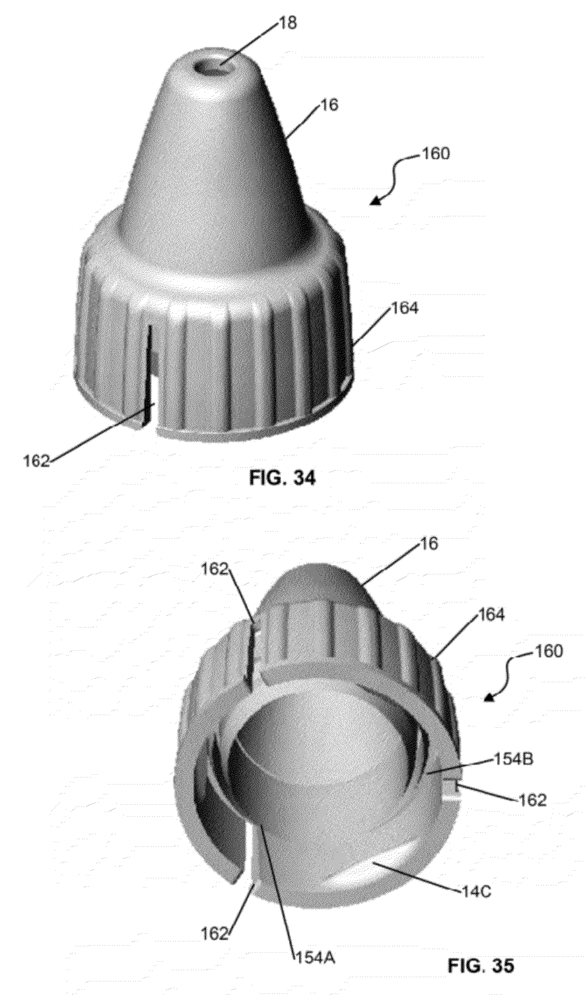

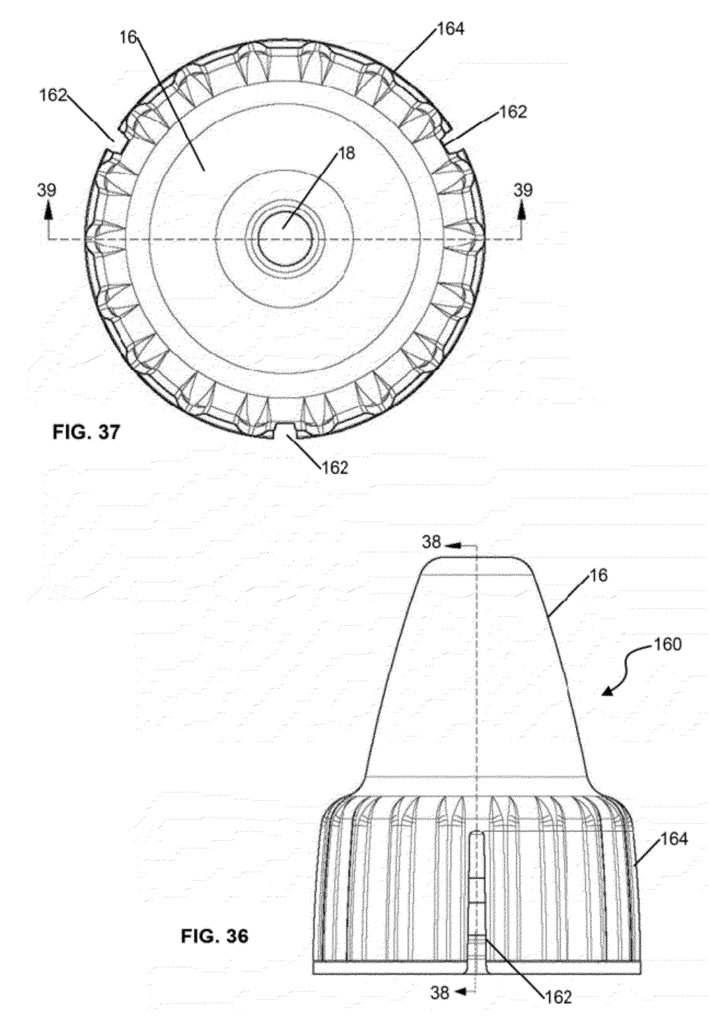

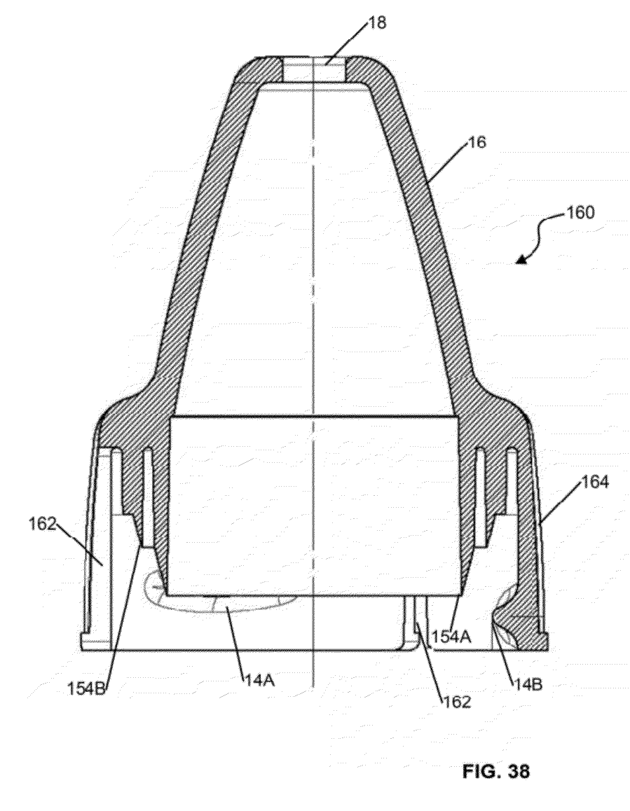

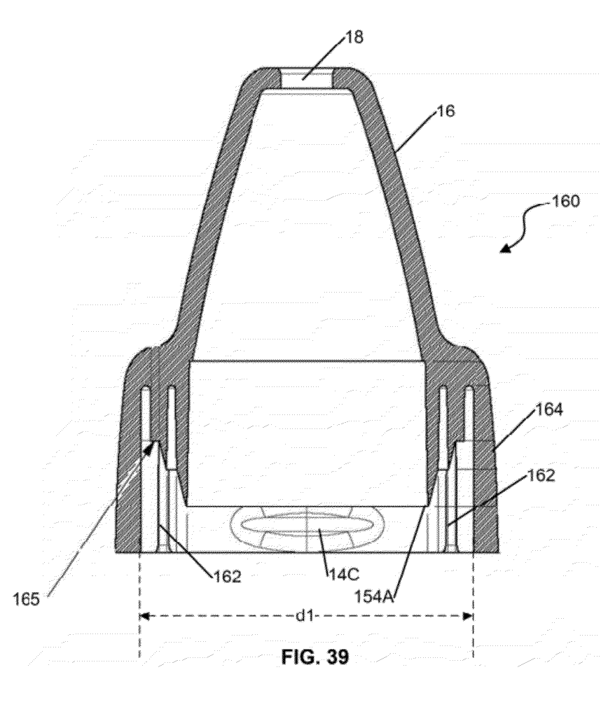

FIGS. 34 to 39 illustrate a modified embodiment of an adapter cap 160 which is similar to cap 150 but designed to fit on larger diameter bottle necks. Some parts of cap 160 are identical to corresponding parts in the first embodiment and the embodiment of FIGS. 28 to 33, and like references numerals are used for like parts as appropriate. Cap 160 is very similar to cap 150 and also has three equally spaced slits or relief slots 162 in connector or neck portion 164. However, slits 162 are longer than slits 151 for increased flexibility, and cap 160 has a dual lip seal 165 with annular lips or rims 154A and 154B (see FIGS. 38 and 39) for engaging inside bottle necks of different diameters, unlike the single lip or annular rim 154 of cap 150 and cap 10 of the first embodiment.

In one embodiment, each slit 162 has a width of the order of 1.9 mm (0.07 inches) and a height of the order of 14.2 mm (0.56 inches), which is more than 75% of the height of connector portion 164 (16.2 mm or 0.64 inches). The lip or rim 154A has a diameter of around 20.5 mm (0.80 inches), while lip or rim 154B has a diameter of around 23.9 mm or 0.99 inches. The inner diameter d1 of connector portion 164 which fits over the outside of the bottle neck is of the order of 28 mm or 1.11 inches, i.e. slightly larger than the inner diameter of the connector portion 152 of the previous embodiment. The outer diameter of connector portion 164 is of the order of 32.5 mm or 1.28 inches. Cap 160 can be attached to the more common plastic water bottles which have an inner neck diameter of 0.85 inches via lip 154A, as well as to less common plastic water bottles with a one inch inner neck diameter via lip 154B, with the longer slits providing more flexibility for fitting the connector portion over the outer surface of the bottle neck. Cap 160 fits many standard plastic water bottles.

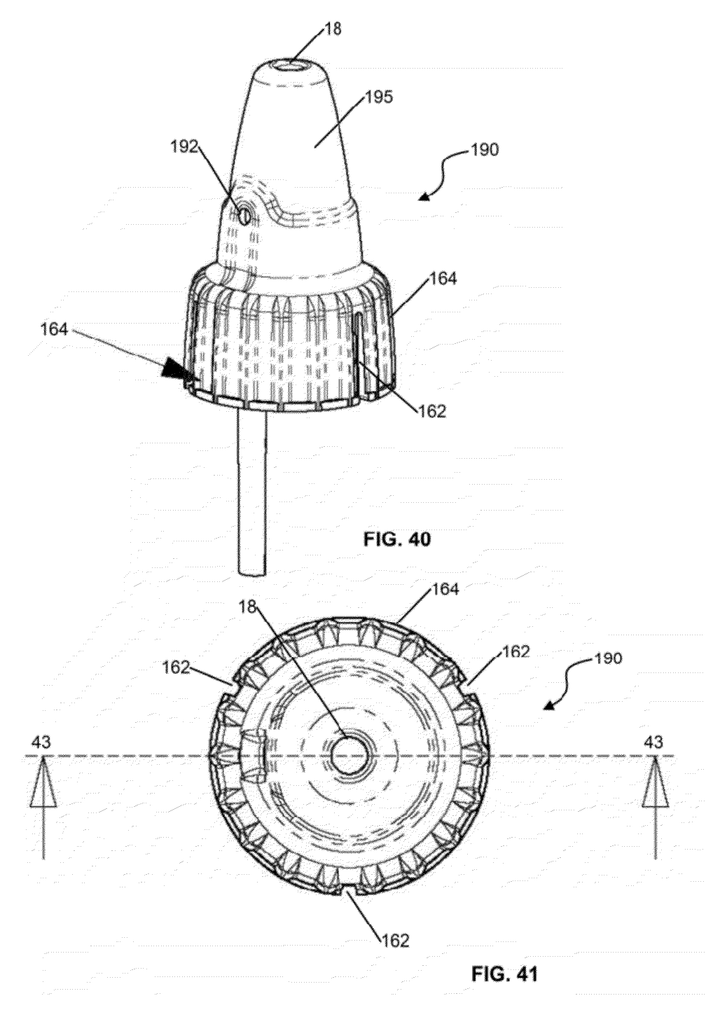

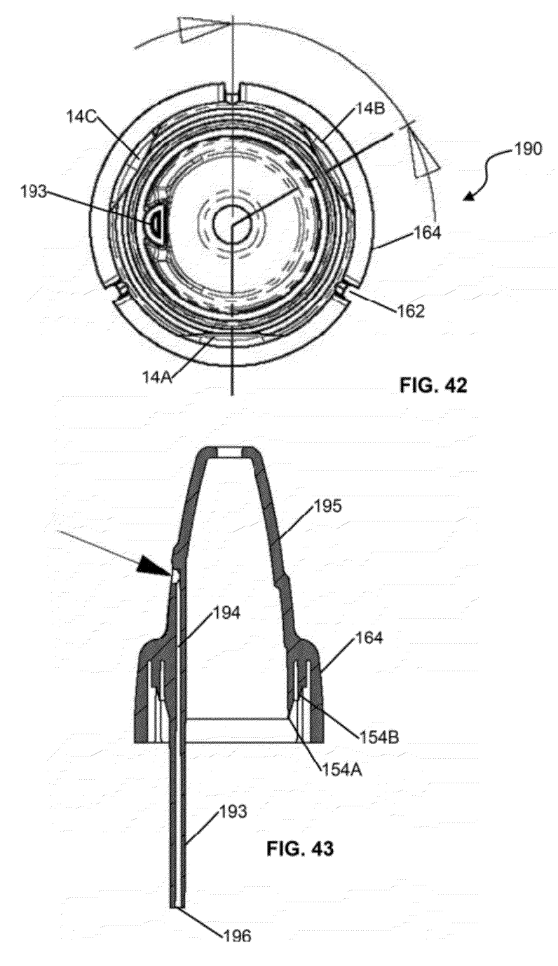

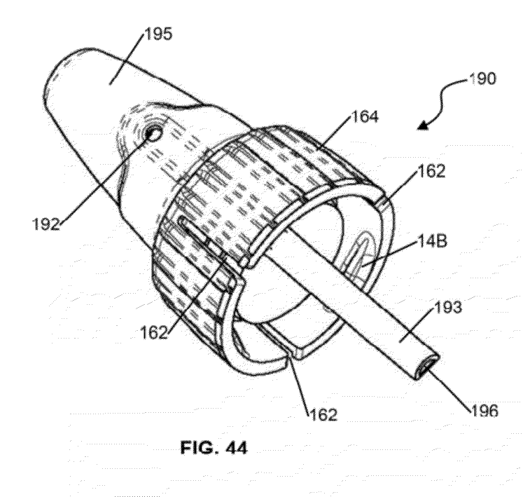

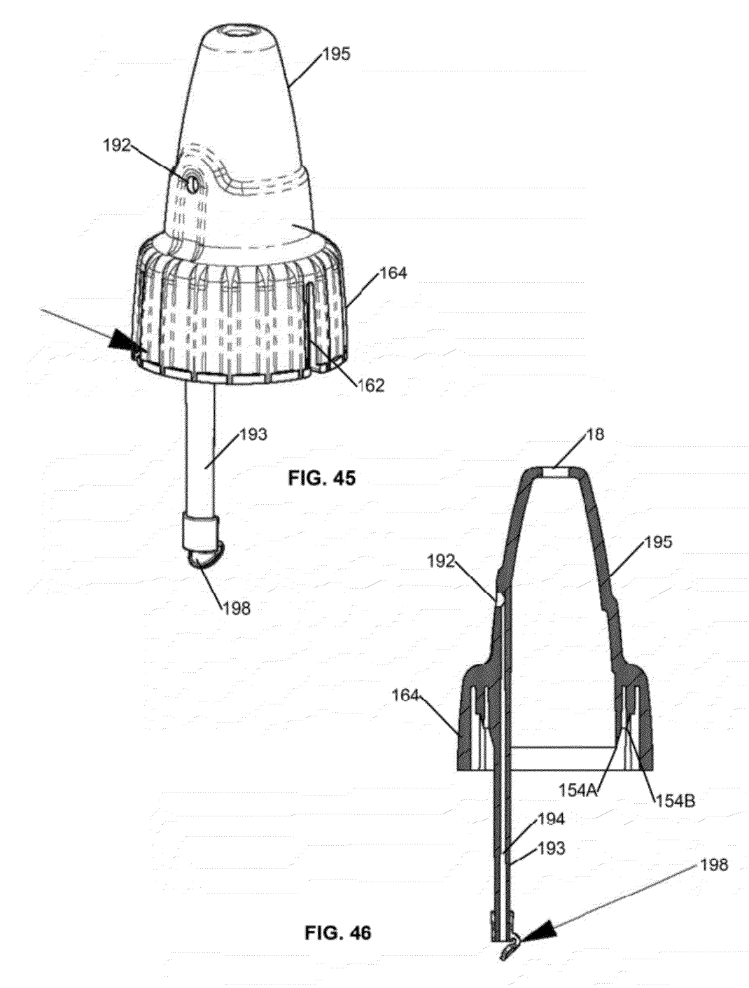

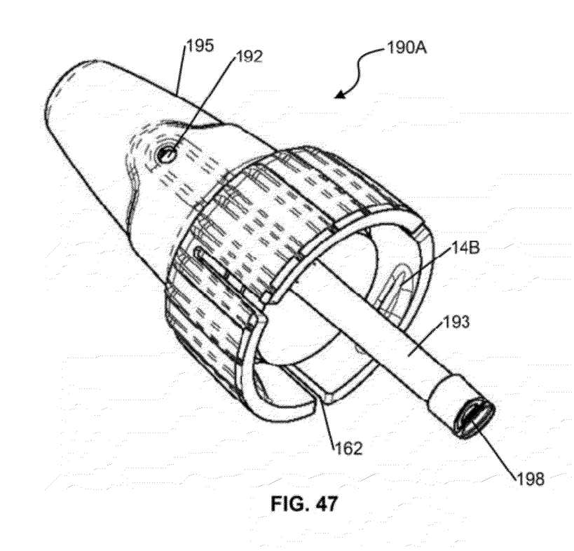

FIGS. 40 to 44 illustrate a cap 190 which is similar to the previous embodiment of FIGS. 34 to 39 but which is modified to include a vent 192 and vent tube 193 extending from vent 192 into an attached bottle, to allow air to enter an attached bottle through passageway 194 and water or saline solution to flow out more easily, as in the embodiment of FIGS. 19 to 22. The cap 190 is otherwise similar to the cap of FIGS. 34 to 39, apart from some dimension changes, and like references are used for like parts as appropriate. As in the previous embodiment, cap 190 has a tapering body or spout portion 195 and a generally cylindrical grip portion or connector portion 164, and three spaced slits 162 are provided around connector portion 164. A dual sealing lip 154A, 154B is provided inside the connector portion 164, as in the previous embodiment. The vent hole 192 is provided in the tapered spout portion 195 of the cap, and vent tube 193 extends downwardly from the vent hole inside the cap and out through the open lower end of the cap to engage inside a bottle when the cap is secured to a bottle neck. Tube 193 has an open lower end 196, but in the modified cap 190A of FIGS. 45 to 47, a check valve or one way valve 198 is located at the lower end of vent tube 193. Valve 198 opens to allow air to enter the bottle, but closes to prevent water from entering tube 193 if the bottle is tilted to allow water to flow out of cap opening 18. Cap 190A is otherwise identical to cap 190, and like reference numbers are used for like parts as appropriate.

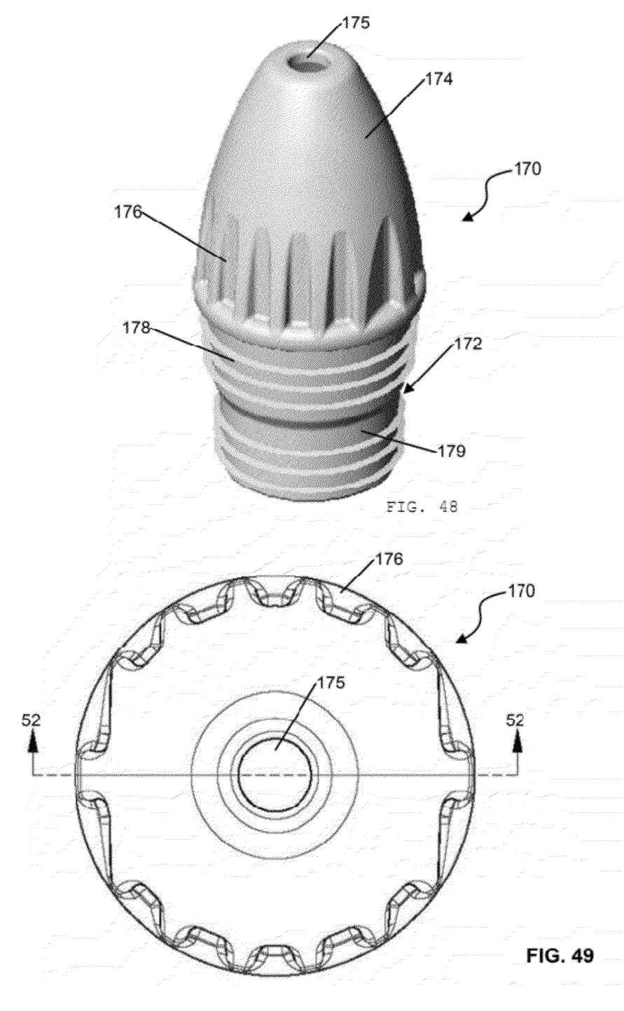

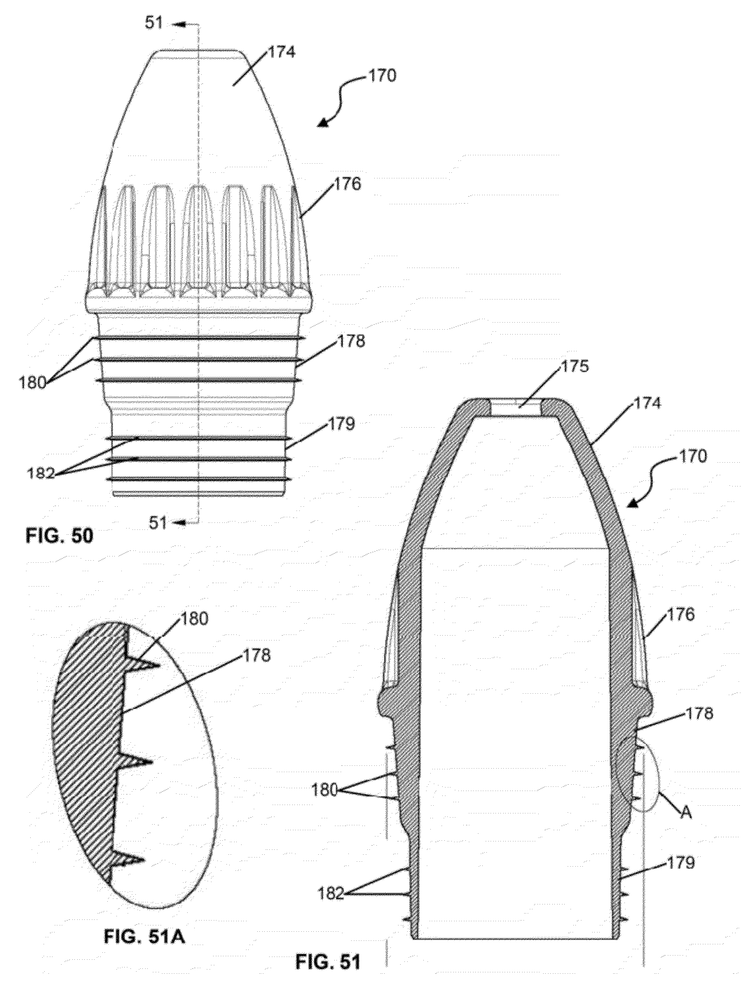

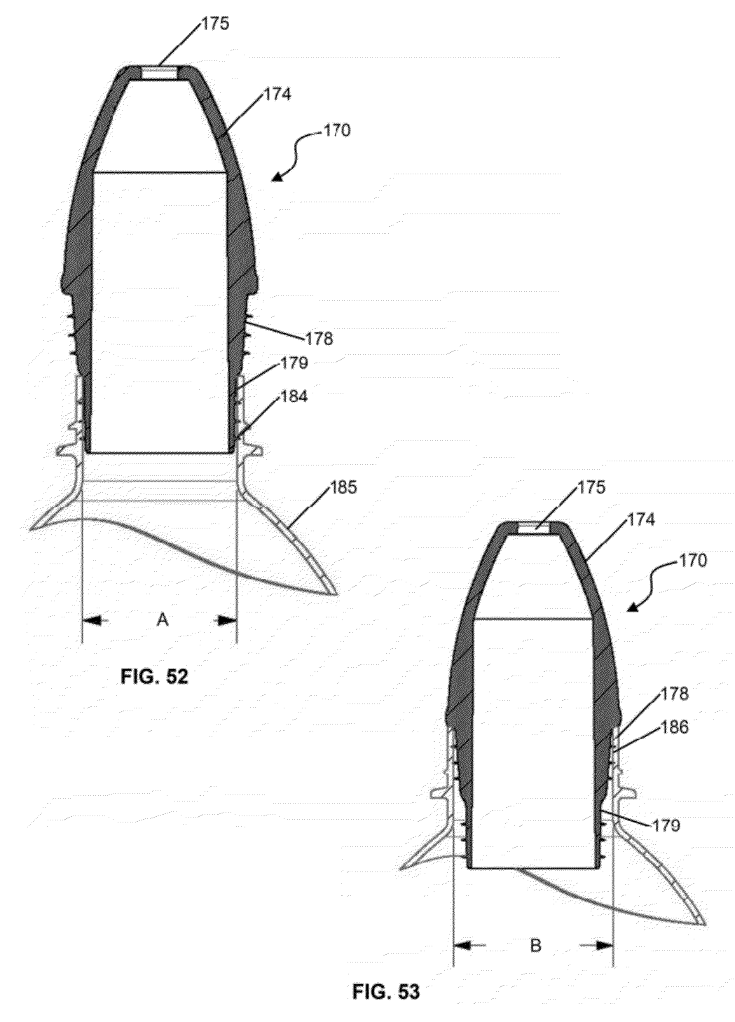

FIGS. 48 to 53 illustrate another embodiment of an adapter cap 170 which is similar to the embodiment of FIGS. 17 and 18, but which has a dual diameter collar 172 replacing single diameter collar 72 of cap 70. Cap 170 has an upper body or spout portion 174 of generally rounded, tapering shape extending up to upper end opening 175, and a gripping portion 176. Dual diameter collar 172 extends downward from the upper body portion, and has a first collar portion 178 extending from upper body portion 174 and a second collar portion 179 of a second, smaller diameter extending from collar portion 178 to the end of collar 172. Each collar portion has spaced, annular outer gripping ribs 180, 182, respectively. The upper or first collar portion 178 is of tapering diameter towards lower collar portion 179, as illustrated in FIGS. 50-53. The lower collar portion 179 is designed to engage in the bottle neck 184 of a first plastic water bottle 185, as indicated in FIG. 52, while the upper, larger collar portion 178 is designed to engage in the larger diameter bottle neck 186 of a different style of plastic water bottle, as indicated in FIG. 53. In one embodiment, smaller diameter collar portion 179 is designed to fit inside the more common plastic water bottles which have an inner neck diameter A of 0.85 inches, while larger diameter collar portion 178 is designed to fit inside the less common plastic water bottles which have an inner neck diameter B of around one inch (typically 0.98 inches).

In one embodiment, the outer diameter of ribs 182 on the lower collar portion 179 was around 0.88 inches so that the ribs are compressed when this portion is forced into a smaller diameter bottle neck as indicated in FIG. 52, gripping against the inner surface of the bottle neck to hold the cap in place. The maximum outer diameter of the uppermost rib 180 of tapering, larger diameter collar portion 178 was around 1.01 inches, so that the ribs are compressed when portion 178 is forced into the larger diameter bottle neck 186 of FIG. 53. The inner cylindrical surface of cap 170 had a diameter of around 1 inch, and the maximum wall thickness of the cap at the junction between body portion 174 and the first tapered collar portion 178 was around 0.16 inches (4.1 mm), while the minimum wall thickness at the lower open end of the cap was around 0.025 inches (0.6 mm.). The height H of annular ribs 180 and 182 was around 0.035 inches, the space S between adjacent ribs was around 0.1 inches or 2.5 mm, and the rib thickness L was around 0.015 inches or 0.4 mm.

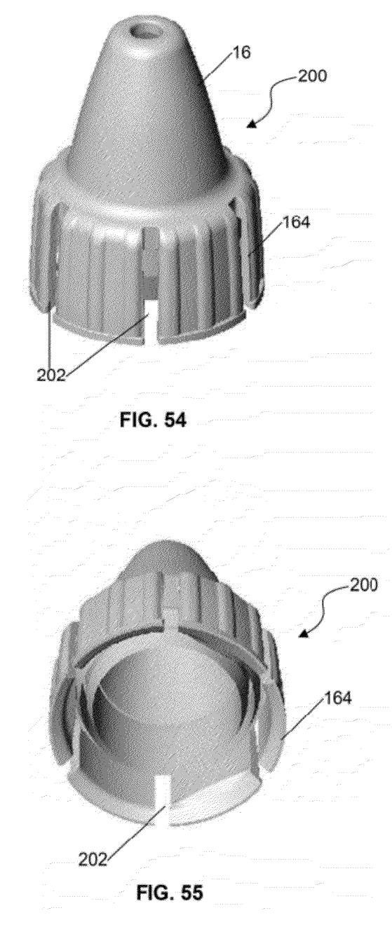

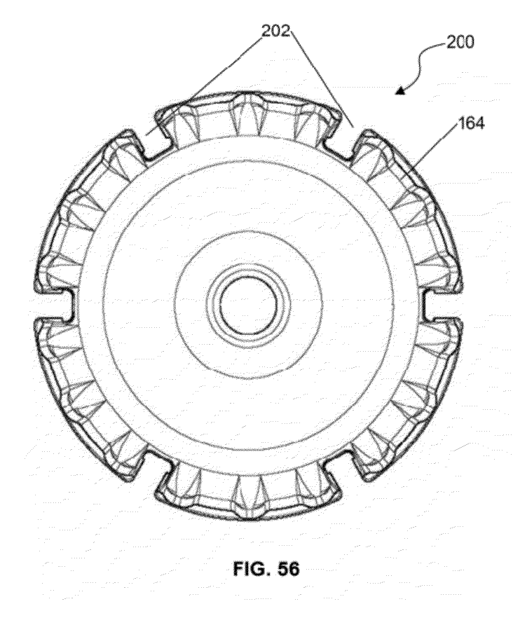

FIGS. 54 to 56 illustrate a modification of the adapter cap of FIGS. 34 to 39. Modified adapter cap 200 has six equally spaced slits or relief slots 202 in connector portion 164, instead of three equally spaced slits or relief slots 162 as in the previous embodiment. This allows for fitting the cap on a larger range of different size bottle necks. Cap 200 is otherwise identical to cap 160 of FIGS. 34 to 39 and like reference numbers are used for like parts as appropriate. Slits 202 may be of approximately the same length as slits 162, and other dimensions of the cap are similar or identical to those of the previous embodiment. Cap 200 has a dual lip seal which may be identical to seal 154A,154B of cap 160, but may have a single lip seal like that of cap 150 in alternative embodiments. The smaller diameter cap 150 of FIGS. 28 to 33 may also be provided with a larger number of equally spaced slits in other embodiments.

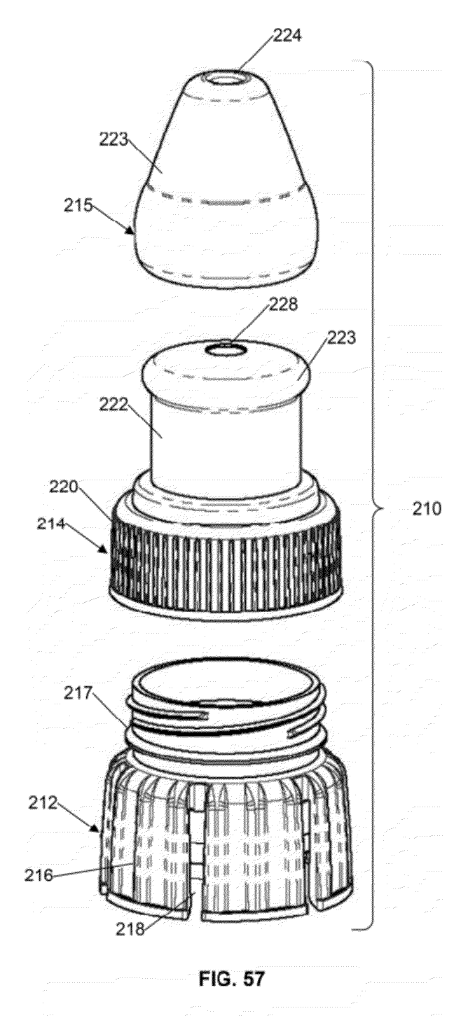

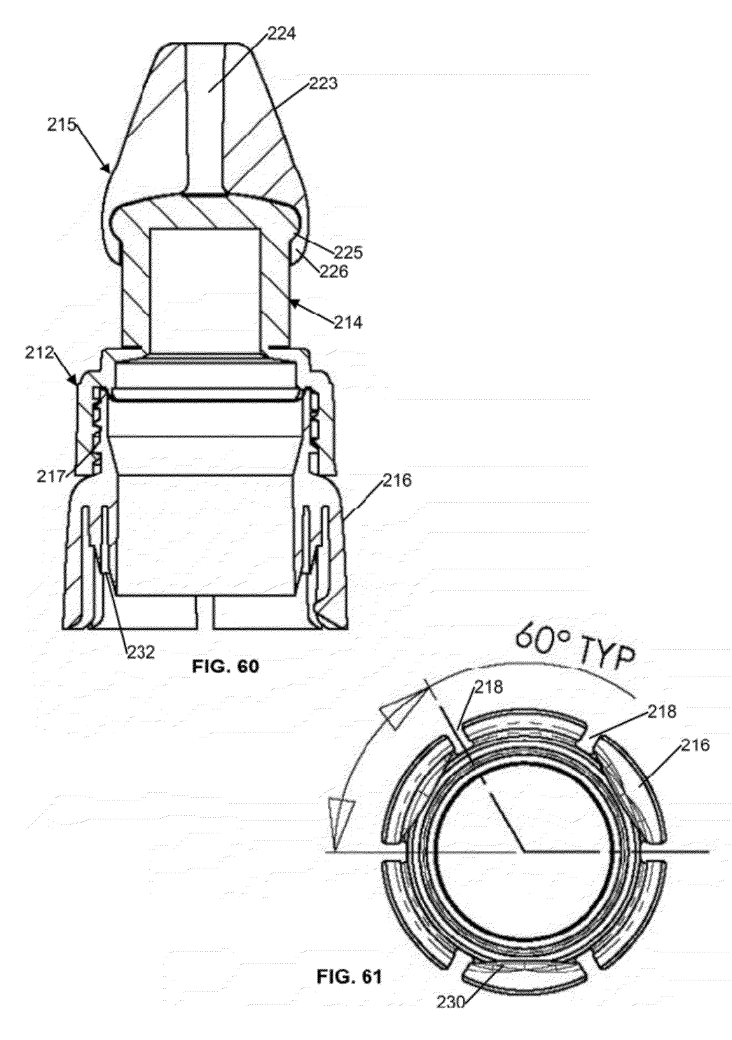

FIGS. 57 to 61 illustrate an adapter cap assembly 210 according to another embodiment which has an adapter sleeve 212 for attachment between the neck of a plastic water bottle and a conventional push/pull bottle cap 214, and a generally frusto-conical nozzle or applicator tip 215 having a tapered spout portion 223 and a lower rim 226 (FIG. 60) configured for releasable snap engagement over the bulbous end portion or annular rim 225 of the push/pull cap.

Applicator tip 215 may be of relatively soft or rubber-like thermoplastic elastomeric material such as SYNOPRENE™ and is softer than the other parts of assembly 210. Tip 215 has an opening 224 at the end of the tip and a through bore extends through the body from the opening 224, as illustrated in FIG. 60. Lower rim 226 extends inwards for releasable snap engagement over the annular upper rim 225 of push/pull cap 214. Push/pull bottle cap 214 is a conventional, off-the-shelf cap with a threaded neck 220 for engagement on a plastic water bottle and a push-pull body 222 which can be pulled out to uncover opening 228 for dispensing water from an attached bottle through the cap, and pushed in to close opening 228 to seal the bottle. This type of push/pull body is known in the field and is therefore not described in detail, and the mechanism of the end opening closure is not shown in detail in the cross-sectional view of FIG. 60.

Adapter sleeve 212 has a connector portion 216 which is similar to connector portion 212 of cap 200 of the previous embodiment, and an externally threaded neck portion 217 extending from an upper end of connector portion 216 in place of the tapering body portion or nozzle of cap 200. As in the previous embodiment, connector portion 216 has six slits 216 and an internal dual lip seal 232 for sealing engagement with the upper rim of a bottle neck, as well as partial threads 230 on its inner surface for engaging over the threads of a large diameter bottle neck. In alternative embodiments, adapter sleeves may be provided with alternative connector portions for connection to the same size bottle neck or smaller conventional bottle necks, such as any of the connector portions of the caps in the embodiments of FIGS. 1 to 53, and with different numbers of slits or no slits. The dual lip seal may be replaced with a single lip seal.

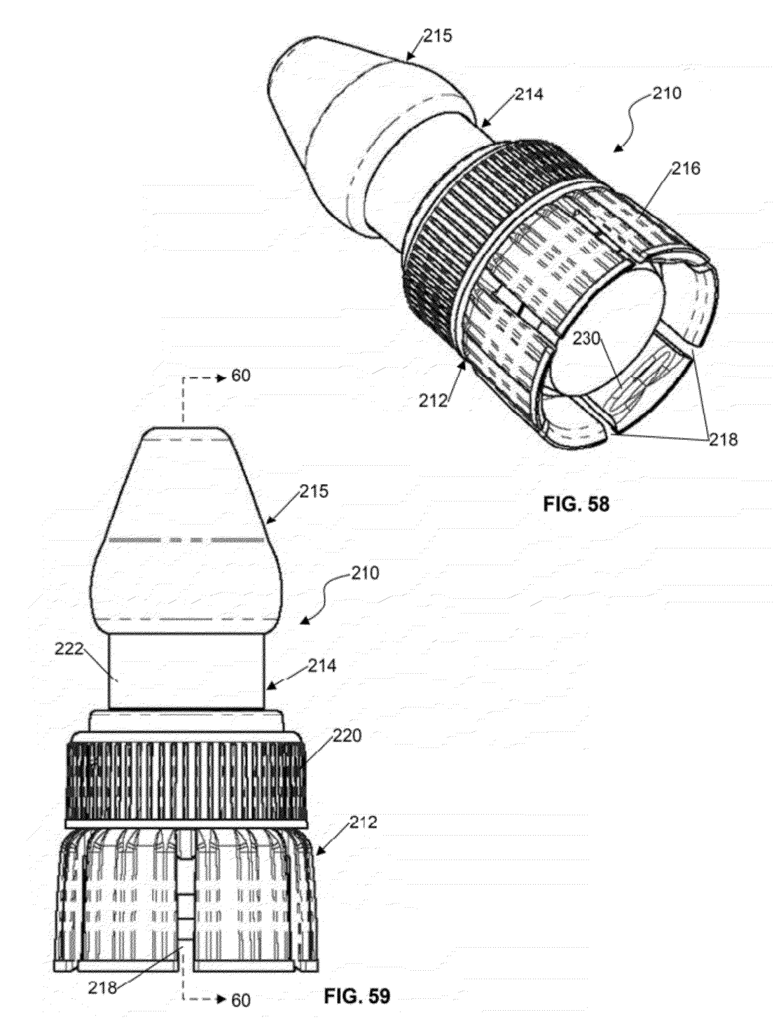

The parts are shown assembled together in FIGS. 58 to 60, and may be assembled before or after adapter sleeve 212 is attached to the neck of a water bottle. As indicated in FIG. 60, connector portion 220 of push/pull cap 214 is threaded over neck portion 217 of adapter sleeve 212, and dispenser tip 215 is snapped onto the upper end rim 223 of body 222 of the push/pull cap. In order to connect the assembly to a water bottle, the existing bottle cap is removed and adapter sleeve 212 is secured to the bottle neck before or after securing push/pull cap 214 to the neck 217 of adapter sleeve.

The advantage of incorporating a push/pull cap component in the adapter cap assembly of FIGS. 57 to 61 is that the bottle can be re-closed after the contents 26 of packet 25 are dispensed into a plastic water bottle as illustrated in FIG. 6 for mixing with the water in the bottle to form a saline solution. As discussed above in connection with FIG. 6, the material added to the water may be a preservative free, pharmaceutical grade salt (or other type of salt), or a mixture of salt with other ingredients. Once the packet is emptied or sufficient dry particulate or powder material 26 has been added to the water, the parts assembled as in FIGS. 58 to 60 are attached to the neck of the bottle and the push-pull body is closed. The bottle is then closed and can then be shaken to mix the salt or salt mixture with the water to form a saline solution, without the user having to close opening 224 with a thumb during mixing. This arrangement is therefore more hygienic. When the user wishes to use the saline solution to wash the nasal cavities, they simply pull out the body 222 of the push/pull cap to uncover opening 228, and tilt the bottle after inserting the soft comfort tip 215 into the nostril, as described above in connection with FIG. 6, allowing the saline solution to be dispensed through adapter sleeve 212 and cap 218, the end opening 228 in body 222 of cap 218, and the passageway 224 through nozzle or tip 215. The bottle can then be closed and re-sealed if desired, without having to remove the adapter cap assembly, simply by pushing body 22 down until the end opening 228 is again closed. The push-pull body therefore also seals the contents of the bottle and keeps both the solution and the tip of the device inserted in the nostril clean if not all the solution is used in one wash. Since most plastic water bottles contain 16 oz of water, the same water bottle with the mixture may be kept and used more than once for nasal irrigation purposes before the bottle is empty and is discarded or recycled.

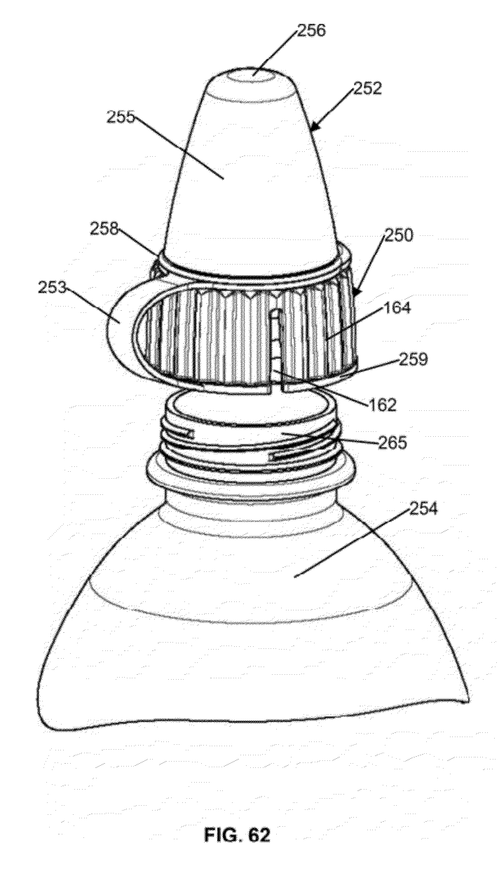

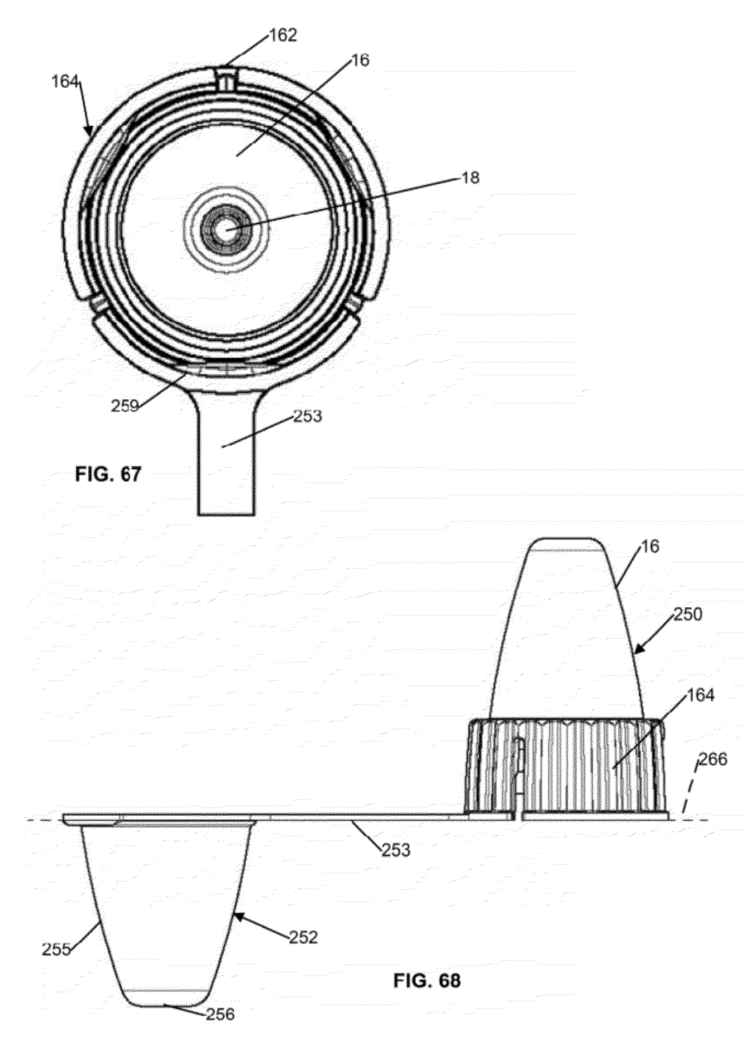

FIGS. 62 to 68 illustrate a modification of the adapter cap 160 of FIGS. 34 to 38 to provide a cap 250 with a mating cover 252 to close the opening 18 in cap 250 when not in use. The cover 252 is attached to the cap 250 via integral strap or tether 253 to keep the cover close to the bottle while dispensing saline solution from bottle 254. The cap 250 is otherwise identical to the cap 160 of FIGS. 34 to 38, except that it has a single lip or annular rim 251 rather than dual lips or rims defining a dual rim seal 165 as in FIG. 39, and like reference numerals are used for like parts as appropriate. Cap 250 may have a dual lip seal 165 as in FIG. 39 in alternative embodiments.

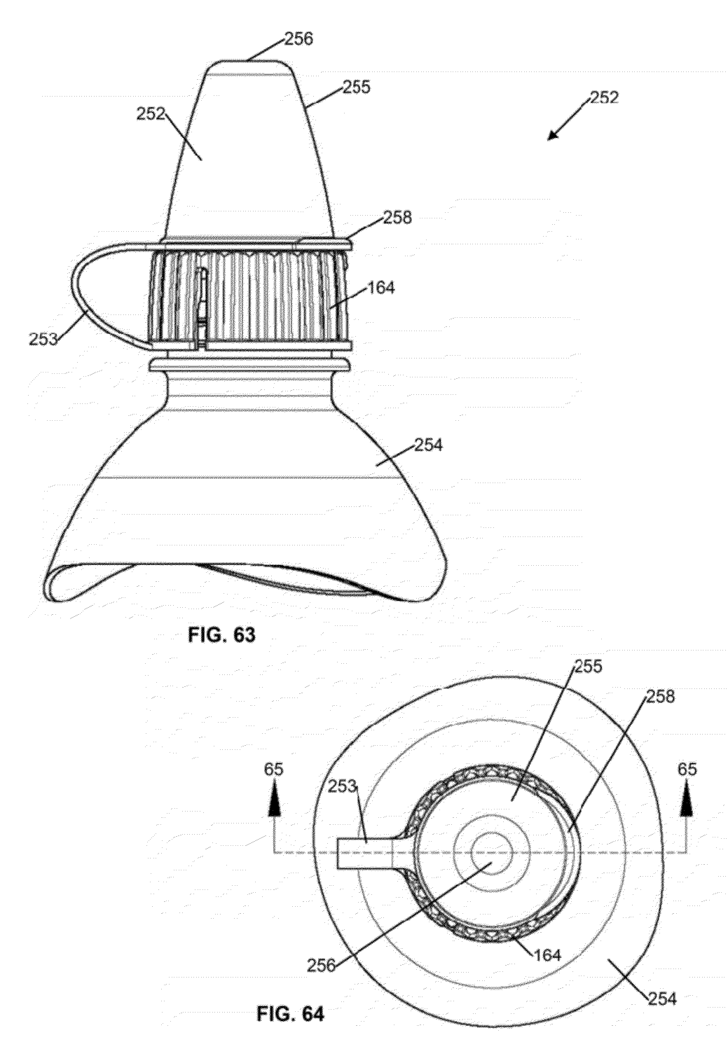

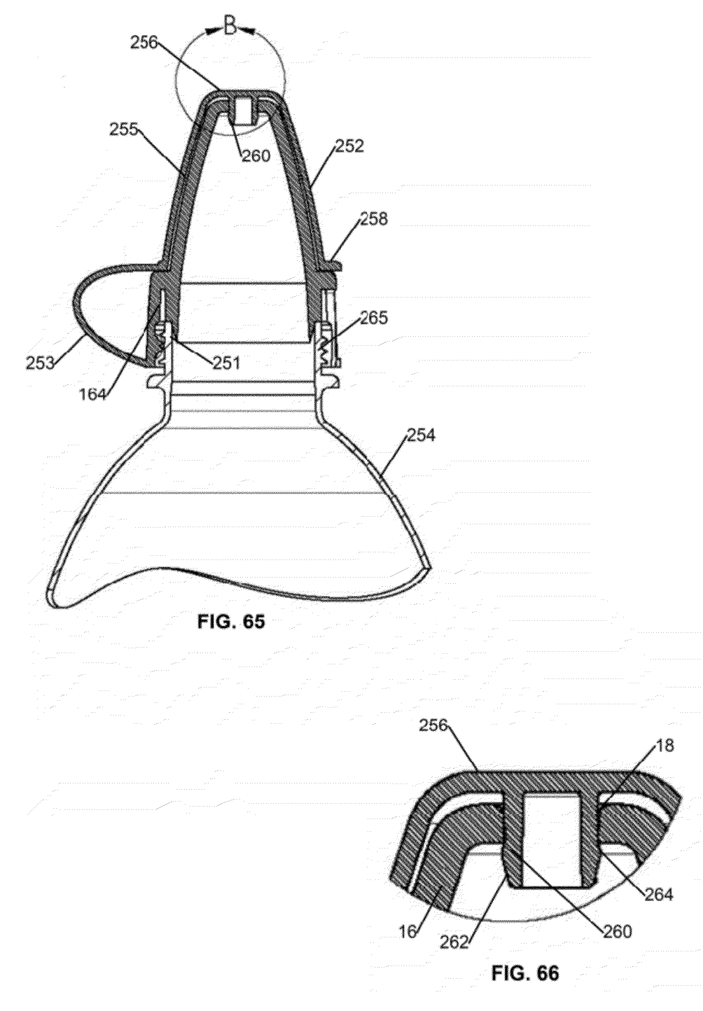

Cover or lid 250 is of the same general frusto-conical shape but slightly larger dimensions than the tapered spout portion 16 of cap 160, and has a tapered skirt portion 255 and closed upper end wall 256 for mating engagement over portion 16 in the closed position, as illustrated in FIG. 65. Cover 250 may have a body of injection molded polypropylene or the like. Tether or strap 252 extends from a lower peripheral rim of portion 255 to the lower end 259 of connector portion 164 of cap 250. The lower rim has an enlarged tab 258 (see FIGS. 64 and 65) located opposite tether or strap 253 for engagement by the user's thumb or the like to detach cover 250 from cap 160.

End wall 256 has a downwardly extending hollow plug or stopper 260 designed for snap engagement in opening 18, as illustrated in FIGS. 65 and 66. As indicated in FIG. 66, stopper 260 has a slight undercut or taper 262 at its lower end, and a slightly raised rib 264 for snap engagement through opening 18.

Cover 252 provides a removable closure for engagement over the spout portion 16 of the cap as needed, for example while the contents of the bottle are being shaken on initial mixing of water with saline solution additives, or when saline solution is not being dispensed. FIG. 68 illustrates the cover and cap in an open condition, and also illustrates the position of these parts when formed in a mold with tether 253 connecting them, with the mold parting line 266 illustrated in dotted outline. When the cover is engaged over portion 16 and snapped in place, the lower rim and finger/thumb tab 258 rest on the shoulder 268 between tapered upper portion 16 and connector portion 164 of the cap, as illustrated in FIGS. 62, 63 and 65. Thus, cover 252 can be used to close cap 250 while the water in the bottle is shaken up to mix with suitable additives as described above to form a saline solution, and also when the bottle containing saline solution is not in use, so that the solution and tip are kept clean during initial mixing and between nasal irrigations. When the user wishes to dispense saline solution in the manner described above in connection with the previous embodiments, they simply push up on tab 258 to release stopper 260 from opening 18, and push the cover off the cap and to one side. The strap 253 keeps the cover close to the bottle while saline solution is dispensed, so that the cover is readily available for placement over portion 16 to re-close and seal the opening while the bottle is not in use.

A similar cover may be provided for any of the caps described in the previous embodiments, with appropriate adjustment of the shape and dimensions of portion 255 for mating engagement over the corresponding tapered portion of the cap. In this way, the user's thumb does not need to be placed over the tip during mixing, and both the tip and contents of the bottles are kept clean during mixing and between uses.

The adapter caps and adapter cap assemblies described above provide an easy and convenient way to convert many conventional plastic water bottles into a nasal wash system for use at home or when away from home. The caps are generally designed to fit most 26-28 mm. plastic water bottles. Bottled water is considered to be a safe, clean, cheap and convenient source of water to use for nasal irrigation, while tap water is no longer recommended for nasal washing or even for washing a nasal irrigation device between uses. Adapter caps can be carried around at all times as well as stored in a bathroom drawer or cabinet for use as needed. A convenient, lightweight kit contains a plurality of adapter caps and a corresponding number of packets containing dry ingredients for mixing with water to produce a nasal washing solution. This can be used at home or when away from home, so that the user has the benefit of using a clean, cheap and convenient source of water for nasal washing at home or when away. Even when camping, hiking or the like in locations having no clean water supply, a user can still use the nasal wash system with bottled water when needed, which may be very helpful in an environment which is dusty, has poor air quality, or air containing a large amount of allergens. The empty water bottle is generally recycled or discarded after use, and the adapter cap is relatively inexpensive and can also be discarded or re-cycled with the bottle, avoiding the need for thorough cleaning prior to the next use, making the system more hygienic and more readily usable when away from home. The adapter cap and nasal washing system using the cap provides an all-natural, scientifically proven, effective method for relieving nasal congestion and soothing irritated nasal passages at all times. The kit is compact and takes up less storage space at home or when traveling than the prior Netipots and squeeze bottle nasal irrigation devices.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly limited by nothing other than the appended claims.

We claim:

1. An adapter cap for converting a standard water or drinking bottle into a nasal washing device, the cap comprising:
a tapered spout portion having a through bore, the spout portion having an applicator extending up to a tip at a first end of the cap and being configured for insertion into a nostril during a nasal washing procedure, the tip having a dispensing opening which communicates with the through bore; and
a hollow connector portion extending up to a second end of the cap and having mating formations configured for releasable attachment to the neck of a plastic water bottle.

2. The cap of claim 1, wherein the spout portion and connector portion are formed integrally as a one piece cap body.

3. The cap of claim 1, wherein the connector portion is of generally cylindrical shape and at least part of the spout portion extending up to the tip is of generally conical shape.

4. The cap of claim 1, wherein the connector portion has an outer surface and an inner surface and the mating formations comprise a plurality of spaced, offset partial threads on the inner surface configured for releasable mating engagement with outer threads on the neck of a plastic water bottle.

5. The cap of claim 4, wherein there are three partial threads located at equal angular spacings about the inner surface of the connector portion.

6. The cap of claim 5, wherein at least one of the partial threads is offset in height on the inner surface relative to the other partial threads.

7. The cap of claim 6, wherein two of the partial threads are located closer to the second end of the cap than said one partial thread.

8. The cap of claim 1, further comprising a plurality of relief slots spaced around the periphery of the connector portion and extending from the second end of the cap.

9. The cap of claim 8, wherein at least three relief slots are equally spaced around the periphery of the connector portion.

10. The cap of claim 9, wherein six relief slots are equally spaced around the periphery of the connector portion.

11. The cap of claim 1, wherein the connector portion is configured for releasable attachment to bottle necks of at least two different diameters.

12. The cap of claim 1, wherein the connector portion has an internal, downwardly extending annular rim spaced from the second end of the cap and configured for engagement inside the neck of a water bottle when the connector portion is releasably secured to the bottle neck.

13. The cap of claim 12, wherein the connector portion has first and second coaxial, downwardly extending annular rims of different diameter, the first annular rim being configured for engagement inside a bottle neck of a first diameter, and the second annular rim being configured for engagement inside a bottle neck of a second, smaller diameter.

14. The cap of claim 1, wherein the connector portion has an outer, ribbed gripping portion.

15. The cap of claim 1, further comprising a cover configured for mating engagement over at least part of the spout portion to close the dispensing opening when the cap is attached to a water bottle and not in use to dispense liquid from the bottle.

16. The cap of claim 15, wherein the cover and spout portion are of matching frusto-conical shape.

17. The cap of claim 15, further comprising a flexible tether which attaches the cover to the connector portion of the bottle, the tether being of predetermined length permitting the cover to be removed from the spout portion to uncover the opening when liquid is to be dispensed.

18. The cap of claim 15, wherein the cover has a closed end wall which extends over the dispensing opening in the spout portion when the cover is engaged over the spout portion, the end wall having a downwardly extending plug configured for snap engagement in the opening in the spout portion.

19. The cap of claim 18, wherein an annular shoulder is located between the spout and connector portions of the cap, the cover has a lower rim which is configured for seating on the annular shoulder when the cover is engaged over the spout portion to close the opening, and the lower rim has an enlarged finger tab configured for engagement by a user to release the plug from the dispensing opening in the tip of the spout portion.

20. The cap of claim 1, wherein the connector portion comprises a plug having an outer surface with mating formations configured for sealing engagement inside the neck of a water bottle.

21. The cap of claim 20, wherein the mating formations comprise a plurality of spaced annular ribs.

22. The cap of claim 1, wherein the mating formations comprise a plurality of spaced retaining tabs at the second end of the cap, the retaining tabs having teeth configured for snap engagement over an annular collar on the neck of a water bottle.

23. The cap of claim 1, wherein the spout portion and connector portion comprise separate parts and the spout portion is releasably secured to the connector portion.

24. The cap of claim 1, wherein the spout portion and connector portion comprise separate parts, the connector portion is configured for connection between the neck of a water bottle and the threaded end of a water bottle cap having a push pull closure movable between a closed position in which a dispensing opening in the water bottle cap is closed and an open position in which the dispensing opening is open for dispensing water from the cap, and the spout portion of the adapter cap has a lower rim configured for releasable snap engagement over the dispensing end of the push pull closure of the water bottle cap, whereby the opening of the push pull closure communicates with the through bore in the open position of the water bottle cap to allow dispensing of water through the connector portion, water bottle cap, and spout portion and out of the dispensing opening of the applicator tip.

25. A nasal passage washing system, comprising:
at least one adapter cap configured for releasable attachment to the neck of a plastic water bottle; and
a packet of salt mixable with water in the water bottle to form a saline solution;
the adapter cap having a tapered spout portion with a through bore and an applicator tip at a first end of the cap, the tip having a dispensing opening which communicates with the through bore, and a hollow connector portion extending up to a second end of the cap and having mating formations configured for releasable attachment to the neck of the water bottle, at least an end part of the spout portion being configured for insertion into a nostril during a washing procedure.

26. The system of claim 25, further comprising a container, a number of adapter caps and an equal number of packets in the container, each packet containing a measured amount of salt for use in forming a nasal saline solution of predetermined concentration when mixed with water in a standard water bottle to which one of the adapter caps is attached.

27. A method of washing the nasal passages of a user, comprising:
removing an existing cap from a plastic water bottle;
adding a measured quantity of a dry saline particulate or powder material to the water in the water bottle;
releasably attaching a connector portion of an adapter cap to the neck of the water bottle;
closing a dispensing opening at the tip of a tapered spout portion of the adapter cap while shaking the bottle to dissolve the saline material in the water to form a saline solution;
opening the dispensing opening and placing at least part of the spout portion into one nostril while leaning one's head to one side and holding the water bottle so that saline solution flows out of the bottle, through the adapter cap, and out of the dispensing opening into the nostril and through the nasal passages;
removing the tapered spout portion from said one nostril;
placing at least part of the tapered spout portion into the opposite nostril while leaning one's head to one side and holding the water bottle so that saline solution flows out of the bottle, through the adapter cap and out of the dispensing opening into the opposite nostril and through the nasal passages; and
removing the tapered spout portion from the opposite nostril and returning the bottle to an upright position.

28. The method of claim 27, further comprising placing a removable cover over the dispensing opening in the adapter cap when the bottle and adapter cap are not being used for nasal washing purposes.

29. The method of claim 27, further comprising placing a removable cover over the dispensing opening in the adapter cap while the bottle is shaken to dissolve the saline material in the water.

30. The method of claim 27, further comprising discarding the bottle and adapter cap when all the nasal solution in the bottle has been dispensed.
